(19) United States
(12) Patent Application Publication
(10) Pub. No.: US 2024/0036042 A1
(43) Pub. Date: Feb. 1, 2024

(54) CLICK CHEMISTRY AMPLIFIED NANOPORE ASSAY FOR ULTRASENSITIVE QUANTIFICATION OF PROTEINS

(71) Applicant: UNIVERSITY OF SOUTH CAROLINA, COLUMBIA, SC (US)

(72) Inventor: CHANG LIU, CHAPIN, SC (US)

(21) Appl. No.: 18/363,341

(22) Filed: Aug. 1, 2023

Related U.S. Application Data
(60) Provisional application No. 63/394,088, filed on Aug. 1, 2022.

(51) Int. Cl. G01N 33/569; G01N 33/543

(52) U.S. Cl. CPC G01N 33/56988; G01N 33/54346; G01N 33/5695; G01N 2469/10; G01N 2333/161; G01N 2333/35

(57) ABSTRACT

Methods disclosed herein are directed to methods of detecting and/or quantifying an antigen. The ultrasensitive Click chemistry Amplified Nanopore (CAN) assay has been engineered for antigen quantification in a biological sample. According to the present disclosure, the CAN assay is based on the combination of an amplified sandwich assay and nanopore sensing. Methods disclosed herein may be incorporated into a point-of-care test device with the necessary accuracy, portability, cost efficiency, and simplicity to fill the diagnostic gaps in high infectious disease burden areas.

### CROSS REFERENCE TO RELATED APPLICATIONS

This application claims filing benefit of U.S. Provisional Patent Application Ser. No. 63/394,088, filed on Aug. 1, 2022, which is incorporated herein by reference.

### GOVERNMENT SUPPORT CLAUSE

This invention was made with government support under Grant No. K22AI136686 awarded by National Institutes of Health (NIH) and Grant No. 2047503 awarded by the National Science Foundation (NSF). The government has certain rights in the invention.

### BACKGROUND

Utilizing nanometer-scale pores present in biological proteins or fabricated from artificial, solid-state membranes, nanopore sensing has shown single-molecule sensitivity and demonstrated excellent accuracy for amino acid identification and nucleic acid sequencing. Nanopore sensing has also displayed great potential for protein analysis and has begun to emerge as a biomarker detection tool for clinical use. Theoretically, the concentration of an analyte can be determined by recording and statistically analyzing the frequency of ionic current blockade events induced by translocation of single analyte molecules through the nanopore under an applied electrical potential. However, in reality, to obtain statistically significant numbers of translocation events in a reasonable measurement time, the concentrations of the analyte must be higher than the nanomolar level. Additionally, different analytes often generate similar signals due to the non-selective translocation of analytes in nanopores and the stochastic nature of the electrical current blockade signals. Therefore, it is difficult to specifically sense ultra-low amounts of biomarkers mixed with a high abundance of interferent molecules in complex clinical specimens such as serum using nanopores without any recognition receptors. Existing nanopore technologies are thus only used to analyze or sequence high-abundance purified analytes, despite their single-molecule level detection capacity. Furthermore, the electrochemical sensing mechanism and the sensing surface's fixed size limit nanopores' utility to only charged macromolecules with sizes comparable to the pore, such as single-stranded DNA (ssDNA).

As such, there is a need for sensitive and specific methods to detect low-abundance proteomic biomarkers in human serum by nanopores.

### SUMMARY

In general, the present disclosure is directed to methods of detecting and/or quantifying an antigen. The method may include a) co providing a sample comprising the antigen; b) incubating the sample with a capture antibody-modified bead and a detection antibody-modified copper-based nanoparticle to form a sandwich structure complex; c) separating the sandwich structure complex from the sample; d) releasing copper ions from the separated sandwich structure complex in an acidic environment; e) coupling a modified nucleic acid molecule and a second molecule comprising an azido functional group in the presence of the copper ions to form a polynucleotide complex, wherein the modified nucleic acid molecule is coupled to the azido functional group via a click reaction; f) non-covalently bonding a probe to the polynucleotide complex to form a polynucleotide probe; g) loading the polynucleotide-probe into a nanopore; h) applying an electrical potential across said nanopore to induce translocation of the polynucleotide-probe complex through said nanopore; and i) detecting an electrical signal associated with the translocation of said polynucleotide-probe complex through the nanopore; wherein a characteristic of the detected electrical signal is indicative of the presence or quantity of the antigen in the sample.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 illustrates the click chemistry amplified nanopore (CAN) assay workflow for quantification of HIV-1 p24 antigen in a biological sample.

FIG. 1A shows a schematic illustration of an α-HL protein inserted into a lipid bilayer membrane with an applied external voltage.

FIG. 1B shows average open pore current values at ±100 mV and the distribution of calculated thicknesses of lipid bilayer membranes (N=50).

FIG. 1C shows continuous raw current traces of DNA probes achieved in different stages of synthesis. Circles, squares, and triangles indicate alkyne modified DNA, DNA-AA, and DNA-AA@CB[6], respectively.

FIG. 1D shows current traces of alkyne modified DNA, DNA-AA, and DNA-AA@CB[6] translocations, respectively

FIG. 1E illustrates a typical multi-level signature signal generated by translocation of a DNA-AA@CB[6] probe. Each signal event is characterized by a pattern with Level 1 and 2.

FIG. 1F shows two-dimensional scatter plots of alkyne modified DNA translocation signals. Top: histograms of MO with Gaussian fitting. Right: histograms of dwell time with exponential fitting.

FIG. 1G shows two-dimensional scatter plots of DNA-AA translocation signals.

FIG. 1H shows average current blockades of Level 1 & 2 in multi-level current events. ****P<0.0001 (Level 1 vs. Level 2 by two-tailed unpaired Student t-test, N 32 100).

FIG. 1I shows cumulative numbers of multi-level events acquired using different independent nanopores over different recording time (total events N>500 min⁻¹).

FIG. 1J shows cumulative numbers of multi-level events acquired using different independent nanopores with different Cu ion concentrations (total events N>500 min⁻¹).

FIG. 1K shows capture rates of DNA-AA@CB[6] multi-level events under increasing trans potentials from 100 mV to 200 mV.

FIG. 1L shows capture rates of multi-level events recorded using five independent nanopores. Data represents mean±SD of replicates with different pores. Data was acquired using 3 M KCl, 10 mM Tris buffer at pH 8.0 and under 160 mV trans potential unless otherwise specified.

FIG. 2A shows a scheme of Cu⁺ ion-catalyzed click reaction between alkynes (R₁) and azides (R₂).

FIG. 2B shows MS characterizations of the substrate (alkyne modified DNA) and the product (DNA-AA) of the click reaction.

FIG. 2C shows MS characterizations of the substrate and the product of the click reaction under different reaction time in absence of Cu⁺ ons.

FIG. 2D shows MS characterizations of the substrate and the product of the click reaction under the same reaction time with different Cu⁺ ion concentrations.

FIG. 2E shows reaction efficiency as a function of reaction time in presence of various Cu⁺ ion concentrations. Reaction efficiency is determined by S_{DNA-AA}/S_{DNA}+S_{DNA-AA}) (S: integrated peak area in mass spectrum).

FIG. 2F shows reaction efficiency as a function of Cu⁺ ion concentrations under 4 h reaction time with linear fit between 0 and 1 mM Cu⁺ ions. Inset shows the theoretical p24 antigen concentration range covered.

FIG. 2G shows correlation between multi-level signal frequency and Cu⁺ ion concentration. Inset shows the correlation in higher range up to 10 mM. Data represents mean±SD of three replicates (total events N>1000 for each data point). Solid line indicates linear regression. Shadow indicates limits of 95% confidence interval.

FIG. 3A shows schematic illustration of two sandwich structures obtained for assays with the traditional route (I) and the amplified route (II).

FIG. 3B shows corresponding ICP-OES results of Cu and Fe ions of both sandwich structures after acid treatment.

FIG. 3C shows representative TEM images of CuONP-Linker and CuONP-Linker-Streptavidin structure.

FIG. 3D shows representative Hydrodynamic size distribution profiles of CuONP-Linker and CuONP-Linker-Streptavidin structures.

FIG. 3E shows ¹H NMR (400 MHz) spectra of reactants (middle and lower) and achieved PEG linkers (upper).

FIG. 3F shows FTIR spectra (left) and thermogravimetric curves (right) of bare CuONPs, Linker, and CuONP-Linker structures.

FIG. 3G shows correlations between multi-level signal frequency and p24 concentration in human serum within the range of 0-46.7 pM (0-1000 pg/mL, final concentration: 0, 0.5, 1, 10, 100, 1000 pg/mL) processed using route I and route II assays, respectively. Insets show the correlations within lower range (0-50 fM) and wide range (0-40 pM). Data represents mean±SD of five parallel experiments (total events N>2000 for each data point, detailed information is shown in Table S1). Solid line indicates linear regression with slopes of 0.315±0.045 (route I, R²=0.97, equation: y=0.590×^{0.315}) and 0.261±0.004 (route II, R²=0.99, equation: y=0.818×^{0.261}). Shadow indicates limits of 95% confidence interval.

FIG. 4A shows quantitative measurements of p24 in clinical samples using the CAN assay, with the clinical diagnosis of each patient. Bar graph data represents mean ±SD of three parallel measurements (total events N>1000 for each sample). Each diamond represents the result from one measurement. The dashed line indicates the LOD of the CAN assay.

FIG. 4B shows cluster map produced from p24 results measured by the CAN assay and ELISA for 118 eligible patients. Darker shading indicates results below LOD.

FIG. 4C shows p24 detection accuracy for the CAN assay and ELISA in different patient groups using VL as reference (Negative: n=11; VL-0: n=33; LOD-30: n=55; Positive: n=19).

FIG. 4D shows average levels of p24 concentrations obtained by the CAN assay for different patient groups. P values were calculated by one-way ANOVA with post-hoc Tukey tests among 3 (black) or 4 (gray) groups. **** indicates P<0.0001, ns indicates no significant difference. For CAN assay, P (Negative vs. VL-0)=0.76, F (11,33)=17.3; P (Negative vs. LOD-30)<0.0001, F (11,55)=101.0; P (VL-0 vs. LOD-30)<0.0001, F (33,55)=101.0; P (Negative vs. Positive)<0.0001, F (11,19)=709.2.

FIG. 4E shows average levels of p24 concentrations obtained by ELISA for different patient groups. P values were calculated by one-way ANOVA with post-hoc Tukey tests among 3 (black) or 4 (gray) groups. **** indicates P<0.0001, ns indicates no significant difference. For ELISA, P (Negative vs. VL-0)=0.32, F (11,33)=126.1; P (Negative vs. LOD-30)=0.44, F(11,55)=109.0; P (VL-0 vs. LOD-30)=0.89, F (33,55) =109.0; P (Negative vs. Positive)<0.0001, F (11,19)=481.7.

FIG. 4F shows ROC curves for p24 detection by the CAN assay and ELISA in HIV and AIDS populations.

FIG. 4G shows correlation between CD4 counts and CAN measured p24 concentrations. h: Correlation between viral loads and CAN measured p24 concentrations.

FIG. 5A shows corresponding raw open pore current traces at ±100 mV without any analytes in both sides of the nanopore.

FIG. 5B shows the I-V curve of a single open α-HL nanopore in the work solution of 3 M KCl, 10 mM Tris, pH 8.0. The slope of the fitted curve was 2.58±0.02 ns.

FIG. 6A shows raw current traces of alkyne modified DNA translocations in α-HL. Data was acquired using 3 M KCl, 10 mM Tris buffer, pH 8.0. Triangles indicate multi-level signals.

FIG. 6B shows raw current traces of alkyne modified DNA-AA translocations in α-HL. Data was acquired using 3 M KCl, 10 mM Tris buffer, pH 8.0. Triangles indicate multi-level signals.

FIG. 6C shows raw current traces of alkyne modified DNA-AA@CB[6] translocations in α-HL. Data was acquired using 3 M KCl, 10 mM Tris buffer, pH 8.0. Triangles indicate multi-level signals.

FIG. 7 shows molecular structures and corresponding contour plots depicting the blockade (MO) vs dwell time for DNA and DNA-AA.

FIG. 8 shows representative time scaled (20 s and 2 s) raw current traces of CB[6] translocations. Data was acquired using 3 M KCl, 10 mM Tris buffer, pH 8.0. The final concentration of CB[6] in the cis side is 0.1 mM.

FIG. 9 shows raw current traces of probe DNA-AA@CB[6] translocations under an increasing positive transmembrane potential from 100 mV to 200 mV. Red triangles indicate multi-level signals.

FIG. 10 shows continuous raw current traces of probe DNA-AA@CB[6] translocations under an increasing negative transmembrane potential from −120 mV to −150 mV.

FIG. 11 shows continuous raw current traces of probe DNA-AA@CB[6] translocations under an increasing positive transmembrane potential from 100 to 220 mV (indicated by different shadow colors). Current leakage can be observed with applied potential above 200 mV.

FIG. 12A shows translocation frequency of multi-level signals of probe DNA-AA@CB[6] with different KCl concentration.

FIG. 12B shows translocation frequency of multi-level signals of probe DNA-AA @ CB[6] with different pHs of the work solution.

FIG. 13 shows Mass spectrometry characterizations of DNA-AA obtained with different Cu³⁰ ion concentrations and various reaction times.

FIG. 14A shows multi-level signal frequency of probe DNA-AA@CB[6] as a function of the incubation time with CB[6].

FIG. 14B shows ¹H NMR (400 MHz) spectra of DNA-AA, CB[6] and DNA-AA @ CB[6]. The binding interaction between DNA-AA and CB[6] complex can be deduced via the chemical shift of protons of the CB[6] molecule from methylene (CH2) groups (5.68 ppm and 5.43 ppm) and tertiary C—H groups (5.64 ppm).

FIG. 15 shows distribution of subpopulations of alkyne modified DNA and DNA-AA obtained with various Cu⁺ ion concentrations characterized by HPLC retention time Corresponding two-dimensional scatter plots of translocation signals of all subpopulations (i.e. alkyne modified DNA, DNA-AA, and DNA-AA @CB[6]); and raw current traces of probe DNA- AA@CB[6] obtained with various Cu⁺ ion concentrations.

FIG. 16 illustrates one step synthesis of biotin-PEG linkers and conjugation of biotin-PEG linkers to CuO nanoparticle surface.

FIG. 17A shows representative time scaled (10 s, 2 s, and 200 ms) raw current traces of probe DNA translocations obtained for human serum samples spiked with 0 pg/mL p24 antigens. Data was acquired using 3 M KCl, 10 mM Tris buffer, pH 8.0. Stars indicate multi-level signature events.

FIG. 17B shows representative time scaled (10 s, 2 s, and 200 ms) raw current traces of probe DNA translocations obtained for human serum samples spiked with 0.5 pg/mL p24 antigens. Data was acquired using 3 M KCl, 10 mM Tris buffer, pH 8.0. Stars indicate multi-level signature events.

FIG. 17C shows representative time scaled (10 s, 2 s, and 200 ms) raw current traces of probe DNA translocations obtained for human serum samples spiked with 1 pg/mL p24 antigens. Data was acquired using 3 M KCl, 10 mM Tris buffer, pH 8.0. Stars indicate multi-level signature events.

FIG. 18A shows FTIR spectra of Cu clusters showing a typical absorption band of the carboxyl group at 1720 cm⁻¹ corresponding to the weak interaction of glycine residues of GSH with Cu clusters. Compared to pure GSH, the characteristic peak of SH at 2521 cm⁻¹ disappeared in GSH-stabilized Cu clusters, suggesting the interaction between thiol group and Cu clusters.

FIG. 18B shows thermogravimetric curves of Cu clusters.

FIG. 18C shows fluorescence spectra of blue and red fluorescent CuNCs in aqueous solution with increasing concentrations from 0.001 to 1 mM/mL.

FIG. 19A illustrates structure of the blue emission fluorescent CuNCs linked immunosorbent assay.

FIG. 19B shows the excitation and emission spectra of blue fluorescent CuNCs in aqueous solution recorded upon excitation at 370 nm with a Xe lamp. Inset shows the photograph of a corresponding sample under UV light (365 nm).

FIG. 19C shows calibration curve of the blue emission fluorescent CuNCs-based p24 assay in human serum within the range of 0.04-46.7 pM (1-1000 pg/mL). Inset shows assay results in the range of 0.04-2.1 pM (1-50 pg/mL).

FIG. 19D shows structure of the red emission fluorescent CuNCs linked immunosorbent assay.

FIG. 19E shows the excitation and emission spectra of red fluorescent CuNCs in aqueous solution recorded upon excitation at 360 nm with a Xe lamp. Inset shows the photograph of a corresponding sample under a UV light (365 nm).

FIG. 19F shows calibration curve of the red emission fluorescent CuNCs-based p24 assay in human serum within the range of 0.04-46.7 pM (0-1000 pg/mL). Inset shows assay results in the range of 0.04-2.1 pM (1-50 pg/mL).

FIG. 19G illustrates schematic of an ELISA based fourth generation HIV-1 testing kit for p24 in human serum.

FIG. 19H shows calibration curve of an ELISA based fourth generation HIV-1 testing kit for p24 in human serum within the range of 0-46.7 pM (0-1000 pg/mL, final concentration: 0, 0.5, 1, 5, 10, 50, 100, 300, 1000 pg/mL). Insets show the correlation within lower range 0-0.2 pM (0-5 pg/mL) and corresponding pictures of ELISA results.

FIG. 20 shows LOD and dynamic linear range (DLR) comparison of CAN assay to previously reported comparable immunoassays.

FIG. 21 shows clinical validation participant disposition flow diagram.

FIG. 22 shows quantitative measurement of p24 in clinical samples using ELISA, with the clinical diagnosis of each patient. The dashed line indicates the LOD of ELISA.

FIG. 23 shows correlation between CD4 percentage and CAN assay measured p24 concentrations.

FIG. 24 shows densitometric analysis of p24 concentrations across different groups classified by CD4 counts (≤200, 201-350, 351-499, ≥500/mm3). P values were calculated by one-way ANOVA with post-hoc Tukey tests. * indicates P<0.05, ns indicates no significant difference. P(≤200 vs. 201-350)=0.55, F (9,21)=253.3; P(≤200 vs. 351-499)=0.73, F (9,15)=286.8; P (201-350 vs. 351-499)=0.99, F (21,15)=286.8; P (≤200 vs. ≥500)=0.03, F (9.62)=97.64.

FIG. 25A shows chemical structure of DNA-A, DNA-AA, and DNA-AA@CB[6]. DNA (5′-CCCCCCCCCCTCCCCCCCCCC) modified with an alkyne moiety on the T base formed DNA-A; a click reaction between the alkyne moiety and azide adamantane formed DNA-AA; non-covalent host-guest interaction between DNA-AA and CB[6] formed a DNA-AA@CB[6] probe.

FIG. 25B shows electrospray ionization mass spectrograms of reactant DNA-A and product DNA-AA of the click reaction.

FIG. 25C shows 1 H NMR (300 MHz) spectra of reactant CB[6] and achieved DNA-AA@CB[6] probes. The host-guest interaction can be confirmed by the chemical shift of protons of the CB[6] molecule from methylene (CH2) groups (5.66 ppm and 5.44 ppm) and tertiary C—H groups (5.61 ppm).

FIG. 25D shows blockade vs. dwell time translocation profiles of DNA-A. Top: histograms of relative frequency of I/I₀ with Gaussian fitting. Right: histograms of relative frequency of dwell time with exponential fitting.

FIG. 25E shows blockade vs. dwell time translocation profiles of DNA-AA. Top: histograms of relative frequency of I/I₀ with Gaussian fitting. Right: histograms of relative frequency of dwell time with exponential fitting.

FIG. 25F shows blockade vs. dwell time translocation profiles of DNA-AA@CB[6]. Top: histograms of relative frequency of I/I₀ with Gaussian fitting. Right: histograms of relative frequency of dwell time with exponential fitting.

FIG. 25G shows a typical characteristic oscillation signal generated by the DNA-AA@CB[6] probe translocating an α-HL nanopore. The signal is characterized by a pattern with 3 levels. Average currents of Level 1, 2, and 3 in oscillation signals were analyzed by one way ANOVA. **** indicates P<0.0001 (Level 1 vs. Level 2, Level 2 vs. Level 3 by multiple comparisons, N=30). Data were acquired using 3 M KCl, 10 mM Tris buffer at pH 8.0 and under 160 mV trans potential unless otherwise stated.

FIG. 26A shows raw translocation recordings of serially diluted ESAT-6/CFP-10 antigens (0 aM, 10 aM, 10 fM, and 10 pM) through single α-HL nanopores; triangles, squares, and circles indicate oscillation signals in different data sets.

FIG. 26B shows capture rate of oscillation signals in different ESAT-6/CFP-10 antigen concentrations. The cumulative numbers of signals were counted every 20 s. Solid lines indicate linear regression.

FIG. 26C shows correlations between oscillation signal frequency and ESAT-6/CFP-10 antigen concentration in human serum within the range of 0.1-10⁶ fM. Inset shows the correlations within attomolar range (0, 10 and 100 aM). Data represents mean±SD of three replicates. Solid line indicates linear regression. Shadow indicates limits of 95% confidence interval. Data were acquired using 3 M KCl, 10 mM Tris buffer at pH 8.0 and under 160 mV trans potential unless otherwise specified.

FIG. 27A shows quantification of ESAT-6/CFP-10 antigen complex in different groups by the nanopore assay. Insets show cases with low concentration ESAT-6/CFP-10. Data represents mean±SD of three replicates. Data were acquired using 3 M KCl, 10 mM Tris buffer at pH 8.0 and under 160 mV trans potential unless otherwise stated.

Repeat use of reference characters in the present specification and drawings is intended to represent the same or analogous features or elements of the present invention.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

In general, disclosed herein are methods of detecting and/or quantifying an antigen. The method may include a) collecting a biological sample comprising the antigen; b) incubating the biological sample with a capture antibody-modified bead and a detection antibody-modified copper-based nanoparticle to form a sandwich structure complex; c) separating the sandwich structure complex from the biological sample; d) releasing copper ions from the separated sandwich structure complex in an acidic environment; e) coupling a modified nucleic acid molecule and a second molecule comprising an azido functional group in the presence of the copper ions to form a polynucleotide complex, wherein the modified nucleic acid molecule is coupled to the azido functional group via a click reaction, wherein a probe is non-covalently bound to the polynucleotide complex; f) loading the polynucleotide-probe complex into a nanopore; g) applying an electrical potential across said nanopore to induce translocation of the polynucleotide-probe complex through said nanopore; and g) detecting an electrical signal associated with the translocation of said polynucleotide-probe complex through the nanopore; wherein a characteristic of the detected electrical signal is indicative of the presence or quantity of the antigen in the biological sample.

Advantageously, methods disclosed herein are useful for detecting ultra-low abundance antigens. The present disclosure overcomes challenges of sensitivity of direct detection of antigens and antibodies through electrophoresis-based nanopore sensing. Herein, an ultrasensitive Click chemistry Amplified Nanopore (CAN) assay has been engineered for antigen quantification in a biological sample. According to the present disclosure, the CAN assay is based on the combination of an amplified sandwich assay and nanopore sensing. To ensure high specificity, a DNA probe is designed to induce a unique translocation signal that is clearly different from signals of other molecules in the assay system. To realize sensitive and specific detection of low-abundance proteomic biomarkers in a biological sample by use of nanopores, methods herein incorporate click chemistry and host-guest chemistry to develop an assay process that identifies proteins by use of customized single stranded DNA (ssDNA) probes. Taking advantage of the built-in catalytic amplification and specific recognition receptors offered by the ssDNA probes, this nanopore-based testing assay exhibits attomolar (aM) level limit of detection (LOD) when applied to quantify an antigen in a biological sample. Methods disclosed herein may be incorporated into a point-of-care test device with the necessary accuracy, portability, cost efficiency, and simplicity to fill the diagnostic gaps in high infectious disease burden areas.

In one embodiment, utilizing methods disclosed herein an antigen in a biological sample may be detected at a LOD of from about 0.1 femtomolar (fM) to about 100 aM, such as from about 1 fM to about 75 aM, such as from about 10 fM to about 50 aM, such as from about 1 aM to about 45 aM, such as from about 5 aM to about 30 aM, such as from about 10 aM to about 25 aM, such as from about 15 aM to about 20 aM, or any range therebetween. Surprisingly, methods disclosed herein can demonstrate a 100-fold higher analytical sensitivity than a fluorescent copper nanocluster (CuNCs) assay and higher analytical sensitivity than a clinically used benchmark ELISA, respectively.

In one embodiment, a biological sample comprising the antigen may be collected. The term “biological sample” as used herein refers to a sample obtained from a subject (e.g., a patient). For instance, a biological sample may include blood, serum, plasma, or tissue. In some embodiments, the antigen can be measured in a biological sample, such as isolated lymphocytes, cerebrospinal fluid, brain tissue, urine, skin, biopsy material, tumor samples, or other tissue samples. It is understood that obtaining a biological sample from a subject refers to taking possession of said biological sample. For instance, the biological sample can be removed from the subject by a medical practitioner. In some embodiments, the biological sample can be analyzed by the medical practitioner or another person (e.g., a lab technician). In one embodiment, the biological sample may be a human serum sample. The biological samples may be collected using serum separator tubes. In one embodiment, the biological sample may be pre-processed, for instance separated from one or more components of the initial biological sample, e.g., via centrifugation, combined with a carrier liquid or other component, e.g., a saline, a buffer, etc., or any other standard pre-examination processing step as is known in the art.

In one embodiment, the sample (either the biological sample as initially obtained or the pre-processed biological sample) may be incubated with a capture antibody-modified bead. For instance, the capture antibody-modified bead may be a magnetic bead such as a capture antibody-modified dynabeads. The capture antibody-modified dynabeads can be utilized herein to immunoprecipitate an antigen from the serum sample.

In one embodiment, the biological sample may be incubated with the capture antibody-modified bead and a detection antibody-modified copper-based nanoparticle to form a sandwich structure complex. For instance, the detection antibody-modified copper-based nanoparticle may be a detection antibody-modified copper oxide (CuO) nanoparticle. The sandwich structure complex may be separated from the sample, e.g., by use of a magnetic field in the case of a magnetic capture bead.

In one embodiment, following separation of the sandwich structure from the rest of the sample, copper ions generated from the CuO nanoparticle may be released from the sandwich structure complex in an acidic environment. For instance, the sandwich structure complex may be combined with hydrochloric acid. The released copper ions may then be available to catalyze a click reaction to couple a modified nucleic acid molecule and a second molecule to form a polynucleotide complex.

In one embodiment, the modified nucleic acid molecule may include, but is not limited to, double-stranded deoxyribonucleic acid (dsDNA), single-stranded DNA (ssDNA), peptide nucleic acid (PNA), single-stranded ribonucleic acid (ssRNA), DNA/RNA hybrid, or double-stranded ribonucleic acid (dsRNA). For instance, the modified nucleic acid molecule may be an alkyne modified DNA. In one embodiment, the modified nucleic acid molecule may be ssDNA-alkyne (ssDNA-A). There is no particular requirement on the size or sequence of the nucleic acid portion of the modified nucleic acid molecule, for instance, a nucleic acid component can have a sequence length of from about 5 to about 30 nucleic acids, with no limitation as to the particular nucleic acids included in the chain Likewise, there is no particular limitation on the alkyne component of the structure, provided that the alkyne group is available for a copper catalyzed azide-alkyne cycloaddition click chemistry cycloaddition reaction. By way of example, an alkyne can be a terminal functionality that is bonded to a polynucleotide via a straight chain C1-C4 alkyl group. Examples of such modified nucleic acid molecules have been described, such as in U.S. Pat. No. 8,193,335 to Carell et al., and U.S. Pat. No. 11,203,612 to Gremyachinskiy et al., both of which are incorporated herein by reference.

In one embodiment, the second molecule may include an azido functional group. For instance, the second molecule may be azido adamantane (1-azidoadamantane). Other examples of suitable azido functional groups and molecules as may be utilized are known in the art such as those described in U.S. Pat. No. 11,203,612, previously incorporated by reference herein. Examples of the second molecule can include, without limitation, 1-Azidoadaniantane, 4-Azidoaniline hydrochloride. 4S)-4-[(1R)-2-Azido-1-(benzyloxy)ethyl]-2,2-dimethyl-1,3dioxolane, NHS-PEG₄-azide, [3aS-(3aα, 4α, 5β, 7aα)]-5Azido-7-bromo-3a,4,5.7a-tetrahydro-2,2-dimethyl-1,3-benizodioxol-4ol, 3′-Azido-3′-2-azido-1-methylquinolinium tetrafluorohorate, 5-Azidopentanoic acid, 4-Azidophenacyl bromide, 4-Azidophenyl isothiocyanate, 3-(4-Azidophenyl)propionic acid. 3-Azido-1-propanainine, 3-Azido-1-propanol.

According to the disclosed methods, the released copper ions can catalyze a click reaction between ssDNA-alkyne and the azido-functionalized second molecule, e.g., an azido adamantane (AA) to form a polynucleotide complex, e.g., a ssDNA-azido adamantane (DNA-AA) complex with amplification.

In one embodiment, a polynucleotide-probe complex may be formed by contacting a probe with the polynucleotide complex. The probe may include, but is not limited to, cucurbit[7]uril hydrate, CB[7], cucurbit[6]uril hydrate (CB[6]), cyclodextrins or a combination thereof. For instance, the probe may be cucurbit[6]uril hydrate (CB[6]). In one embodiment, CB[6] may non-covalently bind the polynucleotide complex to form a host-guest structure of polynucleotide probes (e.g., ssDNA-AA@CB[6]). As used herein, “non-covalent bond” may refer to a hydrogen bond, an ionic bond, a van der Waals interaction, a hydrophobic interaction, a polar bond, a cation-pi interaction, a planar stacking interaction, and a metallic bond.

In one embodiment, the targeted antigen may be detected by loading the polynucleotide probes into a nanopore. For instance, the nanopore may be a α-hemolysin (α-HL) nanopore. Applying an electrical potential across said nanopore induces translocation of the polynucleotide-probe through said nanopore. Translocation of the polynucleotide probes through an α-HL nanopore may produce an electrical signal, such as an oscillation signal, that is distinct from non-specific signals of the modified nucleic acid molecule and/or the polynucleotide complex compared to the polynucleotide probes. For instance, using a Cu+ catalyst, released from CuO, a “guest” molecule (e.g., azido adamantane) may be linked to an alkyne group bonded to a polynucleotide via a thymine, and this thus formed polynucleotide complex could then catch a “host” molecule (CB[6]) in solution to form the host-guest polynucleotide probe. Electrical signal of each molecule, including any modified nucleic acid molecules, polynucleotide complexes and the polynucleotide probes may be measured using mass spectrometry, nuclear magnetic resonance (NMR), or analytical tools utilized by skilled artisans.

Signal frequency generated by the polynucleotide probes and any remaining polynucleotide complexes when translocating through an α-HL nanopore correlates with the amount of copper ion catalyst, which in turn correlates to the concentration of the target antigen present in the initial biological sample. Methods utilized herein provides a versatile biosensing strategy that combines the nanopore's single-molecule sensitivity to a polynucleotide with catalytic amplification, resulting in a unique signal pattern that allows detection and quantitation with high confidence.

Methods disclosed herein may beneficially detect ultra-low concentrations of an antigen in a biological sample. The antigen may be independently selected from the group consisting of a viral antigen, an infectious disease associated antigen, or a combination thereof. In one embodiment, the antigen may be an infectious disease associated antigen including, but is not limited to, a Human Immunodeficiency Virus (HIV) associated antigen, a tuberculosis associated antigen, an influenza virus associated antigen, a meningitis associated antigen, or a combination thereof.

In one embodiment, the antigen may be an HIV associated antigen. For instance, the antigen may be a viral capsid protein p24 antigen.

In one embodiment, the antigen may be a tuberculosis associated antigen. The tuberculosis associated antigen may include, but is not limited to, circulating Mycobacterium tuberculosis (Mtb) antigens. For instance, the tuberculosis associated antigen may be Mtb-derived ESAT-6/CFP-10 antigen complex.

The present disclosure may be better understood with reference to the following examples.

### EXAMPLES

### Materials and Methods

Route I-Conjugation of Detection Antibodies on CuO Nanoparticles

Copper oxide nanoparticles (CuONPs) (1 mg) were dispersed in 1 mL Phosphate Buffered Saline (PBS) by ultrasonication for 10 min and then incubated with detection antibodies (100 μg/mL, 1.31 undiluted anti-HIV-1 p24 antibody [38/8.7.47], Abcam, Cat#: ab9044) for 1 h with vortex (30×g). After centrifugation at 3500×g for 5 min to remove the supernatant, antibody functionalized CuONPs were washed twice and redispersed in 1 mL PBS solution, then blocked with 200 μIL BSA (10% in PBS) for 30 min, and finally stored at 4° C. for further use.

Route I—Immunoprecipitation of p24 Antigens from Human Serum and Conversion to Cu Ions

Capture antibody-coated MBs (20 μL in PBS) were washed with 500 μL assay buffer for three times and dispersed in 500 μL diluted healthy donor serum (human serum: assay buffer=1:1, v/v). To establish a calibration curve, standard samples were made by adding various amounts of p24 antigens to diluted sera and vortexing for 30 min at room temperature to reach final concentrations of 0, 0.5, 1, 10, 100, 1000 pg/mL. Next, antibody-modified CuONPs (200 μL) were mixed with each standard sample and vortexed for another 30 min at room temperature. After forming sandwich structures, MBs were magnetically separated and washed three times with washing buffer. To release Cu ions, sandwich structures were then treated with 200 μL HCl (0.1 M) for 10 min with vortex, and washed with 200 μL washing buffer for 5 times. All supernatants were collected and concentrated to 40 μL for constructing probe DNAs.

Route II—Conjugation of Detection Antibodies to Streptavidin

The conjugation experiment follows a simple and rapid procedure provided along with the Streptavidin Conjugation Kit (Lightning-Link) which targets primary amine groups (e.g., lysines). Briefly, streptavidin modifiers and detection antibodies (100 μg, 1.31 mg/mL, undiluted anti-HIV-1p24 antibody [38/8.7.47], Abcam, Cat#: ab9044) were mixed and incubated for 3 h at room temperature without light. The resulting solution was then quenched for 30 min and stored for future use without purification.

Route II—Synthesis of Trithiol-PEG-Biotin Linkers

Biotin-PEG was prepared by adding 200 μM 4-arm-PEG-thiol solution (Laysan Bio, MW: 10,000 Da) to 200 μM biotin-PEG-maleimide solution (Laysan Bio, MW: 5000 Da). The mixture was incubated on a shaker for 24 h at room temperature, followed by filtration using a 10,000 Da cutoff filter.

Route II—Conjugation of Biotin-PEG on CuO Nanoparticles

CuONPs (1 mg) were dispersed in 1 mL PBS, and then incubated with 10 mL biotin-PEG linker solution on a shaker for 24 h at room temperature. After the incubation, assay buffer was added to bring the final NaCl concentration to 0.05 M. After an hour, the NaCl concentration was raised to 0.3 M using 5 M NaCl, and the mixture was incubated with shaking at room temperature for an additional hour. The resulting mixture was aliquoted into low retention tubes and centrifuged at 3500×g for 10 min at room temperature. After removal of the super-natant and two additional washes with DI water, biotin-PEG-CuONPs were resuspended in 1 mL assay buffer and stored at 4° C. until further use.

Route II—Immunoprecipitation of p24 Antigens from Human Serum and Conversion to Cu Ions

Biotin-PEG-CuONPs were linked with avidinylated detection antibodies by mixing for 1 h, and then collected by centrifugation at 3500×g for 5 min at room temperature. After removal of the supernatant and two additional washes with deionized (DI) water, the probes were resuspended in 1 mL PBS solution, then blocked with 200 μL BSA (10% in PBS) for 30 min, and stored at 4° C. until further use. The immunoprecipitation protocol in Route II is the same as in Route I, except that detection antibodies were labeled with biotin-avidin linked CuONPs for amplification of the conversion.

Preparation of DNA-AA@CB[6] Probes

Conjugation between DNA-Alkyne (sequence: 5′-CCCCCCCCCCT* CCCCCCCCCC-3′, T* indicates alkyne-modified thymine; Sangon Bio-technology Co. Ltd.) and AA was carried out using Cu²⁺ catalyzed click chemistry. In general, 3 μL DI water, 4 μL HEPES (100 mM) buffer, 3 μL alkyne-functionalized DNA (100 μin DI water), 4 μL AA (200 mM in acetonitrile), 3 μL ascorbic acid (20 mM), were mixed with 4 μL Cu²⁺ eluent from immunoprecipitation assays (40 mM copper nitrate for characterization samples). The reaction was incubated on a shaker for 4 h at room temperature before termination by adding 4 μL EDTA solution (100 mM). The product (DNA-AA) was purified in batches by centrifugation using Micro Bio-spin P6 columns. Finally, 15 μIL CB[6] aqueous solution (5 mM) was added to DNA-AA and incubated for 4 h. The resulting DNA-AA@CB[6] probe solution was stored at 4° C. for further use.

Kinetic Study of the Cu+ Ion Catalyzed Click Reaction

Different Cu⁺ ion concentrations (0-10 mM) and various reaction times (10-720 min) were adopted to investigate the click reaction efficiency. Final reaction mixtures were subjected to mass spectrometry analysis before incubation with CB[6]. Concentrations of species were determined by peak area integration.

Single-Channel Current Recording

Fabrication of α-HL nanopore sensors. Briefly, 1,2-Diphytanoyl-sn-glycero-3-phosphocholine was used for self-assembly of a synthetic lipid bilayer across an aperture (diameter: 200 μm) on a 25 μm-thick Delrin wall that divided a planar bilayer chamber into two compartments: cis and trans. Both compartments contained 1 mL of work solution. Electrical potential was applied to the trans side using Ag/AgCl electrodes with a Planar Lipid Bilayer Workstation (Warner Instruments) and slowly ramped up to examine the stability of the membrane at ±200 mV. The membrane capacitance was maintained at 160-180 pF with various voltage bias values throughout each experiment. A small amount (-0.05 μg) of α-HL protein were added to the cis compartment while the trans voltage was changed to +100 mV to drive a single protein into the lipid bilayer. After a stable α-HL protein was inserted and con-firmed by an open nanopore current, analytes were added to the cis chamber (grounded), and the ionic current through the pore was recorded under a bias of 160 mV. All experiments were carried out at 25±2° C.

Data Collection and Analysis

Ionic current recordings were collected using a patch clamp amplifier (Clampex, version 11.0.3, Warner Instruments) with a built-in high- pass filter with a corner frequency of 5 kHz. Signals were digitized by a Digidata 1440 A analog-to-digital converter (Molecular Devices) at a sampling frequency of 100 kHz and processed by pClamp software (version 11.0, Molecular Devices). Each sample was measured at least three times using independent nanopores. The raw data was analyzed using an in-house MATLAB (Version R2019a) based algorithm to find the current blockade and the dwell time of each eligible signal event, which are two commonly used parameters for identifying different analytes. The current blockade (i.e., residual current) that represents the capture of single molecules and their translocation through the nanopore is defined as MO (I: average current measured with the analyte inside the pore; IO: the average baseline value in the absence of analytes). Dwell time (i.e., duration) represents the effective interaction time between the nanopore and a single analyst. For the quantification of biomarkers, the frequency of multi-level sig- nature events generated by translocation of probe DNAs were determined by manual inspection to the raw data. Clampfit (version10.7), OriginPro (version 9.0), GraphPad Prism (version 9.0), were used for data analysis, histogram construction, curve fitting, and graph presentation. Python (version 3.7) modules Matplotlib and Seaborn's bivariate kernel density estimator were used for scatter plots and contour plots.

Statistics and Reproducibility

For each of the nanopore-related results, the number of replicates or statistic events was mentioned in the corresponding figure caption or supplementary tables. Recorded current traces with complete blocking of the nanopore (I/I₀=1) were excluded from the statistics for accurate signal capture rate calculation. No data were further excluded from the analysis. Clinical information was blinded until all experiments and p24 quantification were completed.

### Results

Nanopore Characterization and Translocation Behaviors of DNA Probes.

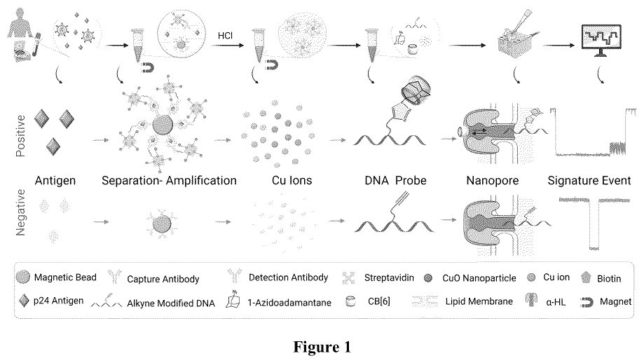

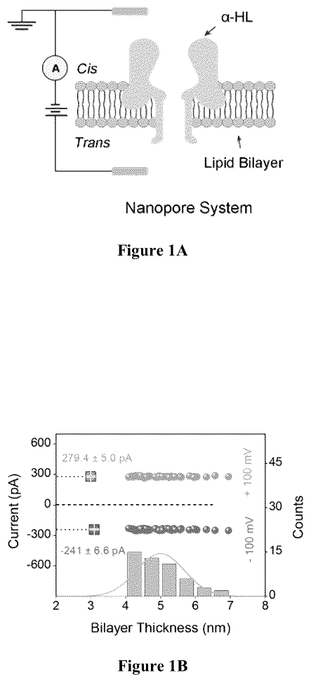

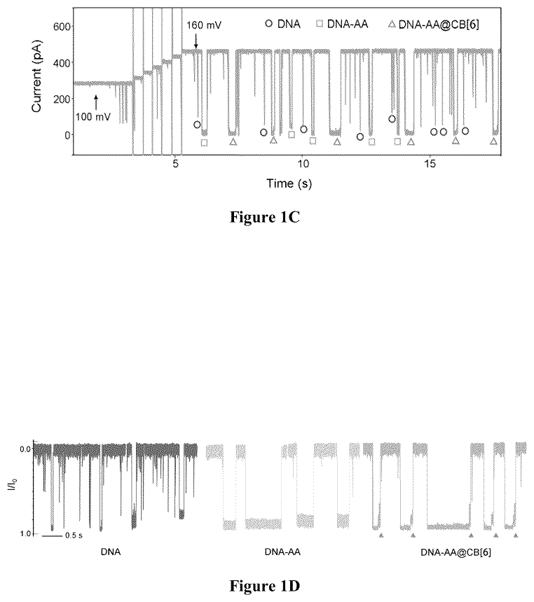

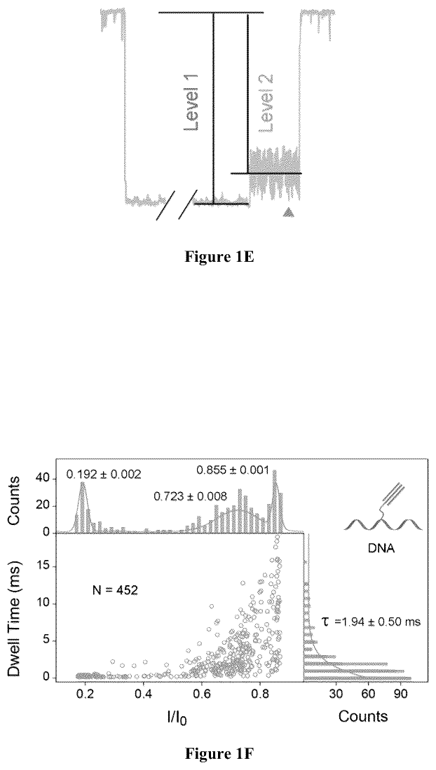

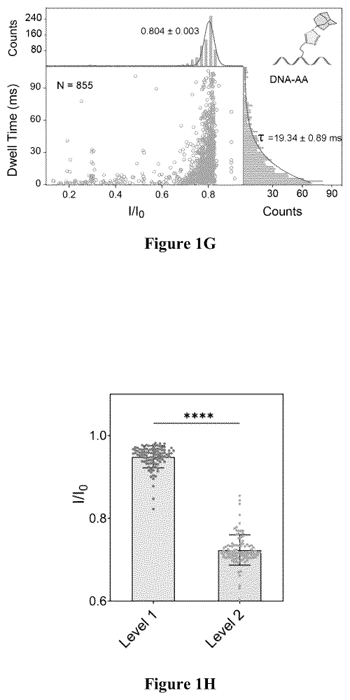

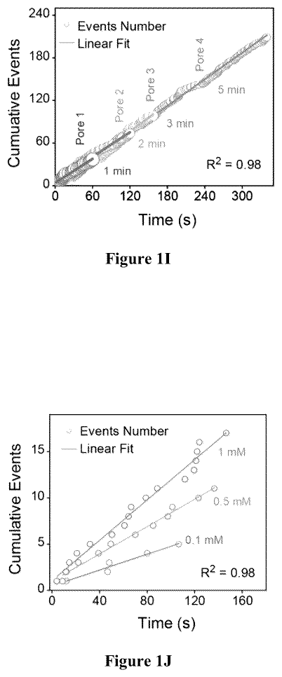

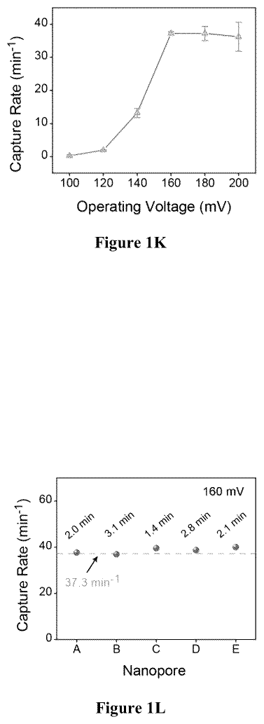

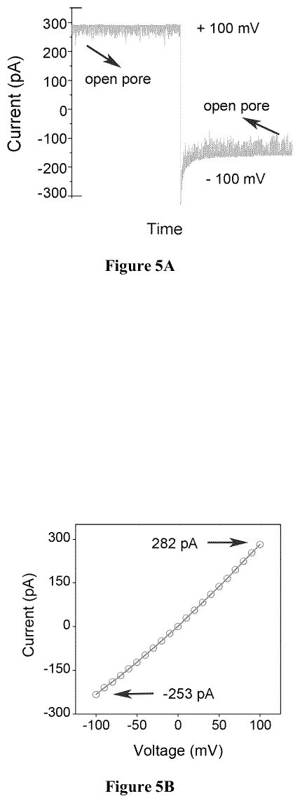

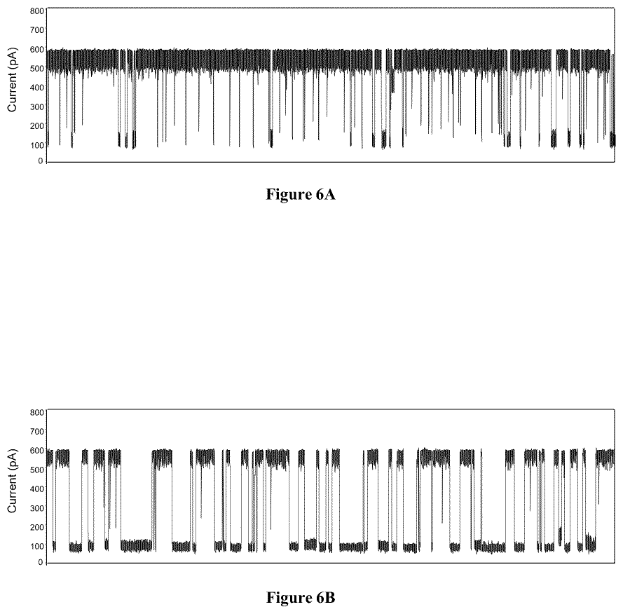

The abovementioned modifications to the DNA probes can endow enhanced sensitivity and specificity for nanopore sensing. For characterization, DNA probes achieved in different stages of synthesis, including alkyne modified DNA, DNA-1-Azidoadamantane (DNA-AA), and DNA-AA@cucurbit[6]uril hydrate (DNA-AA @ CB[6]), were first analyzed for their translocation behaviors in α-hemolysin (α-HL) nanopores, respectively. DNA-AA molecules were obtained through a click reaction of 1-Azidoadamantane (AA) to alkyne modified DNAs in the presence of Cu ions, and DNA-AA@CB[6] complexes were further obtained by a host-guest reaction between DNA-AA and CB[6]. Prior to analyzing the DNA probes, the conductivity of an open α-HL nanopore in the lipid bilayer membrane was probed using a single channel conductance setup (FIG. 1A). Upon insertion of a single α-HL nanopore into the membrane from the cis side, an instant current increase can be observed as shown in the current trace under either a positive or a negative voltage bias (FIG. 5A). Notably, the insertion orientation of the α-HL in the lipid bilayer is pivotal for observing signature current events of DNA probes. A properly inserted α-HL pore is characterized by larger ionic current under a positive trans voltage than it is under a negative trans voltage, i.e., 279.4±5.0 pA under +100 mV, and −241±6.6 pA under −100 mV. The suitable thickness of a bilayer membrane for insertion is ˜5 nm, corresponding to 160-180 pF capacitance (FIG. 1B, calculation details shown in SI). The electrolytic conductance was examined under a ramping voltage to ensure that the conductivity can afford efficient translocations of DNA probes through the α-HL nanopore to induce a large number of blockades to the ionic current (FIG. 5B). When the applied trans voltage is above 160 mV, signals of each subpopulation of the DNA probe synthesis (alkyne modified DNA, DNA-AA, and DNA-AA@CB[6]) can be efficiently observed in the raw current traces (FIG. 1C).

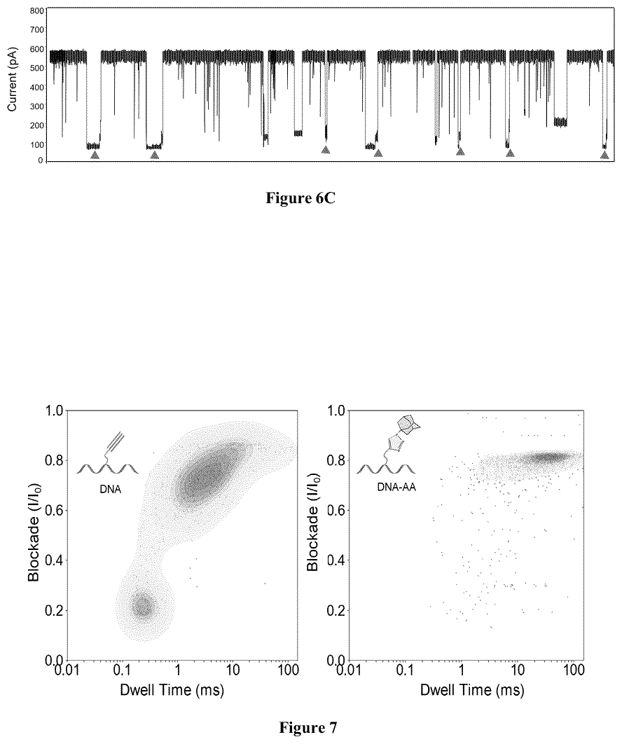

Representative current traces of alkyne modified DNA, DNA-AA, and DNA-AA @ CB[6] (FIGS. 1D and 6)) clearly show differences between these signals induced by the structural evolution of the DNA probes. In comparison with alkyne modified DNA and DNA-AA, the DNA-AA@CB[6] signal has an unique pattern that is characterized by a long deep blockage (Level 1) induced by the translocation of the DNA backbone, and a short oscillation (Level 2) induced by the dissociation of the CB[6] inside the nanopore (FIG. 1e). This pattern is distinctly identifiable from signals of other molecules in this system, which is utilized in this study to ensure highly specific detection. The sensitivity of the detection is afforded by the conversion of p24 antigen to the probe through a DNA click reaction. To quantitatively characterize the evolution of DNA probes, current blockage events of products at each step of the click reaction were statistically analyzed for their current blockade (No) and dwell time (τ). As shown in FIG. 1F, alkyne modified DNA causes distinct blockage events with blockades centered at 0.855±0.001. Observed nonspecific spikes I/I₀ at 0.723±0.008 and 0.192±0.002) can be attributed to occasional random DNA collision with the nanopore or background noise.

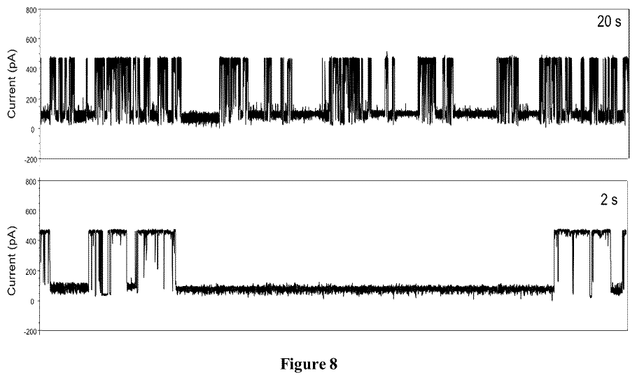

After the click reaction, the product DNA-AA was found to have a blockade profile (0.804±0.003) similar to that of the alkyne modified DNA, with significantly reduced number of nonspecific spikes. However, its mean dwell time was significantly increased from 1.94±0.50 ms to 19.34±0.89 ms (FIG. 1G, P<0.0001, TDNA vs. TDNA-AA by two-tailed unpaired Student t-test). A slight overlap between the contour profiles of alkyne modified DNA and DNA-AA (FIG. 7) could be attributed to the false signals caused by the returning of trapped analytes from the nanopore vestibule to the cis solution without translocation. Although the introduction of CB[6] to DNA-AA was not able to further change its current blockade and dwell time profile, the DNA-AA@CB[6] structure causes a two-stage translocation as previously discussed, that results in a unique signal with two levels of current blockade at 0.948±0.027 and 0.723±0.037 (FIG. 1H) to afford specific and reliable recognition of probe signals from other signals. Excessive free CB[6] in the solution cannot generate multi-level signature signals, thus will not affect its specificity to the DNA-AA@CB[6] probe (FIG. 8).

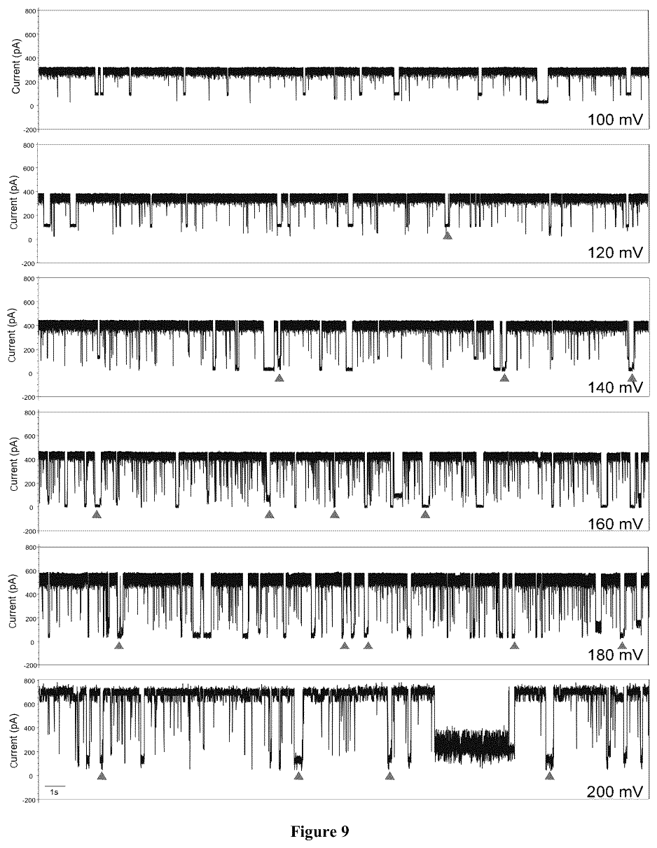

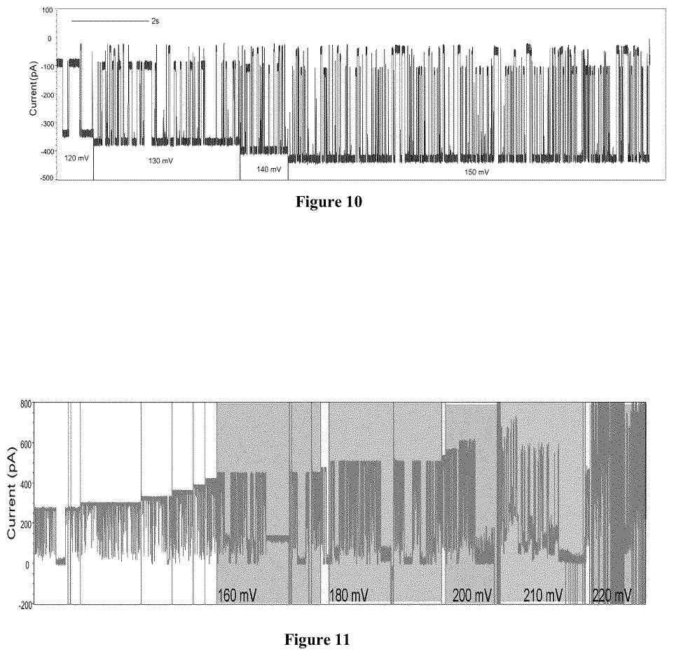

In the capture rate (i.e. translocation frequency) study, standard DNA-AA@CB[6] probe samples were tested using independent nanopores. Events induced by DNA-AA@CB[6] were extracted by observing their multi-level and oscillation characteristics and counted. Cumulative counting of the multi-level signature events shows the same increase rate of event numbers over different recording times (1, 2, 3, 5 minutes) and across four different pores for the same sample (FIG. 1I), which allows capture rate calculation by dividing the multi-level event number over the recording time. While the total single-molecule event rate (alkyne modified DNA, DNA-AA, and DNA-AA@CB[6]) remains consistent at >500 min⁻¹, positive correlation was observed between the capture rate of multi-level events and the Cu ion concentration used to form DNA-AA. The Cu ion concentrations tested (0.1, 0.5, 1 mM) were anticipated to cover most clinical samples (FIG. 1j). The DNA-AA@CB[6] capture rate is also closely related to the applied voltage: less than 2 min⁻¹ multi-level events was observed with 120 mV or lower applied voltage, but it rapidly increased to a maximum of ˜40 min⁻¹ when the applied voltage was ramped up to 160 mV or above (FIG. 1K and FIG. 9). While no multi-level signature event was detected with an increasing negative voltage (FIG. 10), these results indicate that a higher positive voltage can promote the dissociation of the DNA-AA@CB[6] complex and reduce the possibility of DNA escaping back to the cis side. However, high voltage can also weaken the stability of the lipid bilayer membrane, which is shown by the increasing standard deviation and current leakage (FIGS. 1K and 1L). In this study, 160 mV operating voltage and 2-minute recording time was employed to optimize the sample-to-answer time while maintaining membrane stability as well as consistent capacitance (160-180 pF) and interpore capture rate (FIG. 1I).

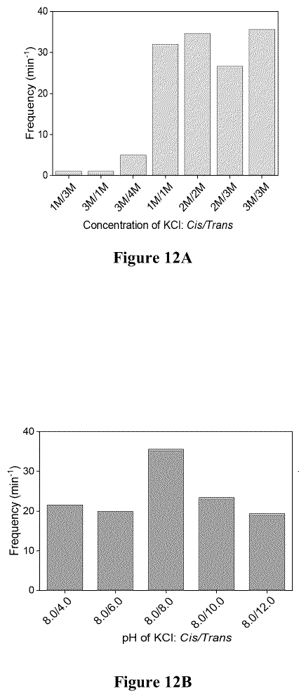

The capture rate of the DNA-AA@CB[6] probes also strongly depends on the electrochemical environment of the nanopore system, which plays a key role in improving the detection sensitivity. To this end, the effects of various electrolyte concentrations and pH of the working solution to the frequency of multi-level signature events were studied. Although concentration gradient was found to increase translocation of linear macromolecules, the results show that optimal multi-level signal frequency can only be achieved using balanced cis and trans work solutions (i.e. with the same electrolyte concentration and pH), and that any disruption of the balance could result in decreased signal frequency (FIG. 12). Therefore, 3M KCl work solution (10 mM Tris, pH 8.0) in both cis and trans sides were employed for all following experiments.

Cu⁺ Ion Concentration Dependent Catalytic Click Reaction.

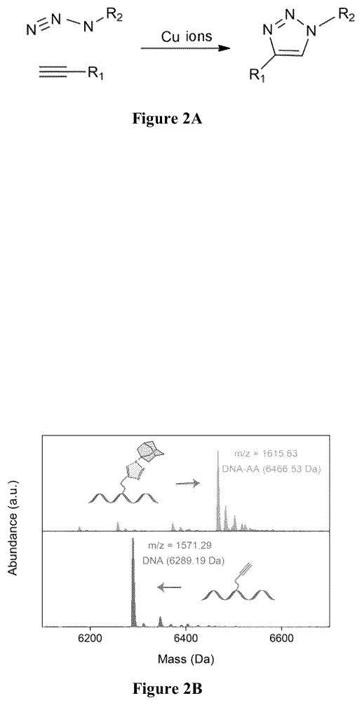

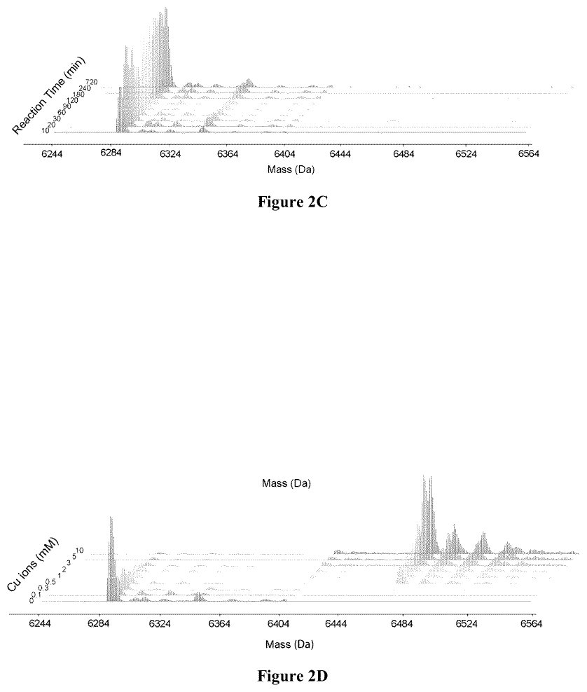

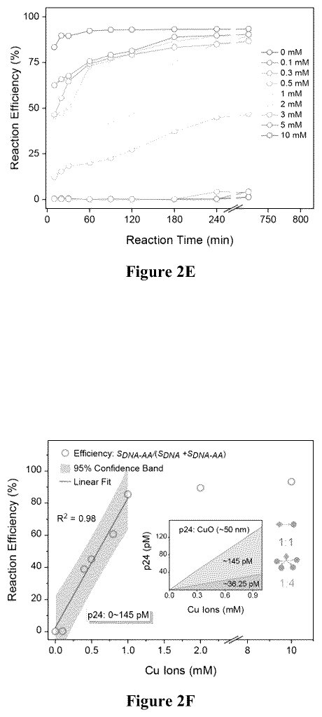

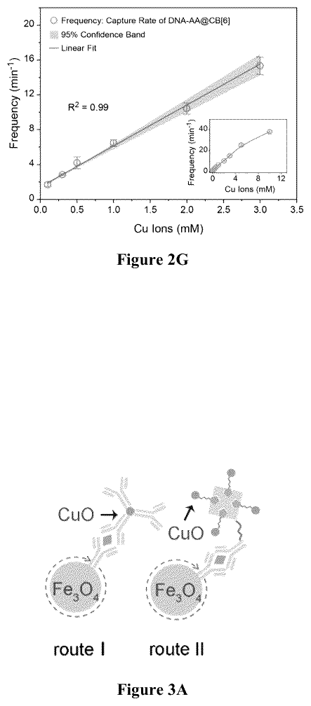

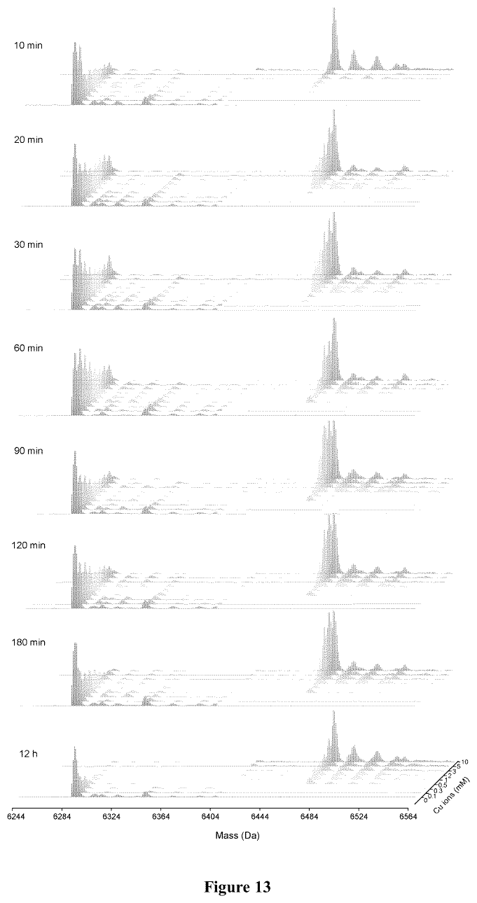

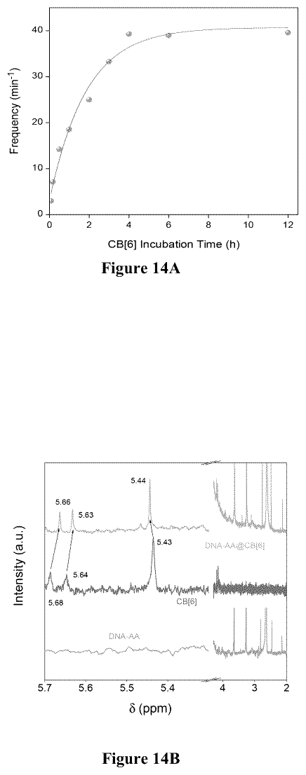

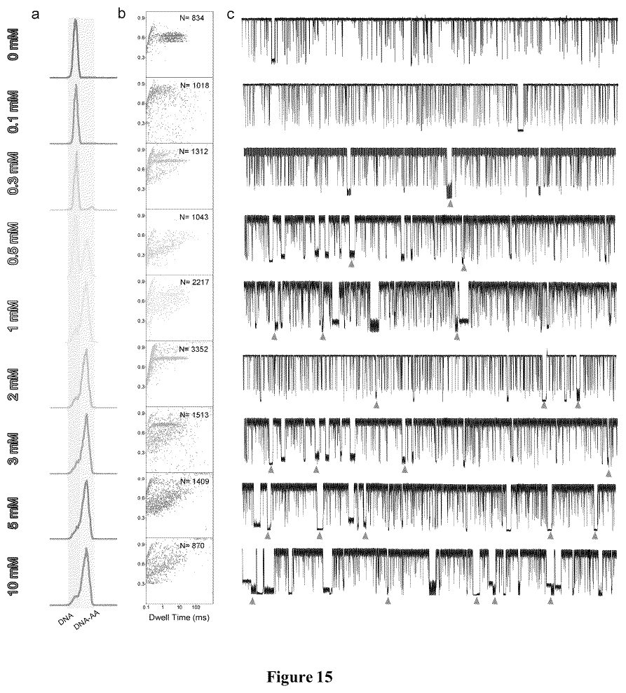

Further optimization was focused on the click reaction between azide and alkyne, which is an essential step in the formation of DNA-AA@CB[6] probes. This reaction can be effectively catalyzed under ambient conditions by Cu⁺ ions (FIG. 2A), which were obtained by sodium ascorbate reduction of Cu²⁺ ions released from CuONPs. Mass spectrometry (MS) confirms the transformation of alkyne modified DNA to DNA-AA after the click reaction (FIG. 2B). The reaction yield is quantitatively related to by Cu⁺ ion concentration with virtually no by-products. No DNA-AA signal can be detected in absence of by Cu⁺ ions with extended reaction time up to 12 h (FIG. 2C). The concentration study indicates increasing DNA-AA signals when by Cu⁺ ions were increased from 0 to 10 mM (FIG. 2D). Further kinetic study shows that the yield in this reaction system is also positively correlated to reaction time under various by Cu⁺ ion concentrations (FIG. 2E and 13). In nanopore measurements of DNA-AA@CB[6] formed with excessive CB[6], an optimal incubation time of 4 h was concluded for maximizing multi-level signal frequency (FIG. 14A). To eliminate the reaction time effect and achieve optimal catalyst-product linearity, click reaction time was limited to 4 h by EDTA termination in all following studies. Although the reaction efficiency plateaued at 10 mM of by Cu⁺ ions, a good linear relationship was obtained between 0 and 1 mM by Cu⁺ ions (FIG. 2F). Theoretical p24 concentrations corresponding to this range are 0-145 pM and 0-36.25 pM for 1:1 and 1:4 p24 antigen-CuONP ratios, respectively (Inset of FIG. 2F), to cover quantification needs in most clinical scenarios (0.42-33.3 pM). While the by Cu⁺ concentration dependency of DNA-AA formation was clearly characterized using HPLC (FIG. 15A) and MS, nanopore measurements, on the other hand, cannot completely separate subpopulations of alkyne modified DNA and DNA-AA translocation events due to signal similarity (FIG. 15). Upon introduction of CB[6] to the click reaction product, the host-guest interaction between CB[6] and DNA-AA can be deduced via nuclear magnetic resonance (NMR) results showing the chemical shift of protons of CB[6] molecules (FIG. 14B). Under these optimized reaction conditions, DNA-AA@CB[6] products formed under various by Cu⁺ ion concentrations were measured by nanopore. Multi-level signal frequencies showed an excellent linear correlation (R²=0.99) with by Cu⁺ ion concentrations used to fabricate DNA-AA@CB[6] probes (FIGS. 2G and S15C).

Optimization of the CAN Assay.

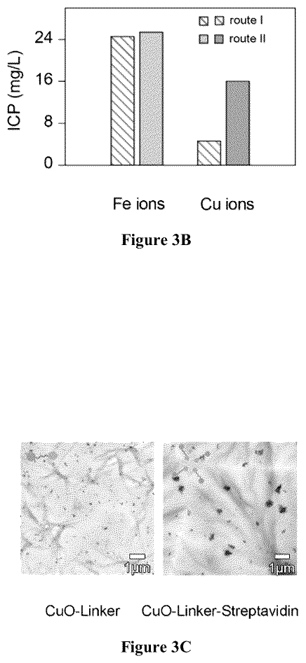

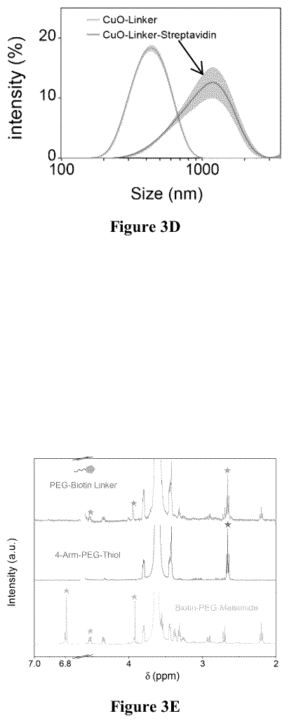

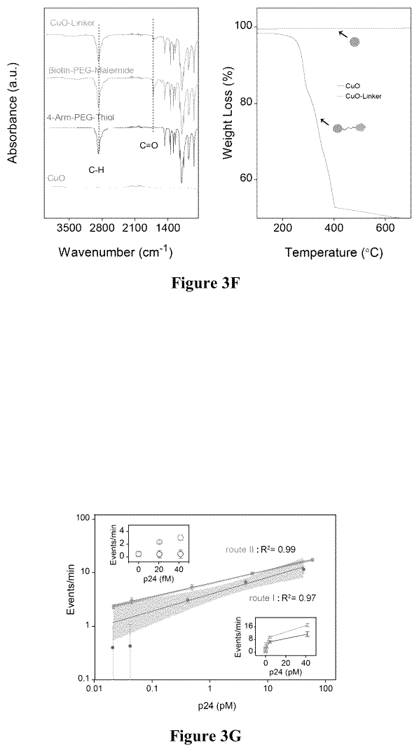

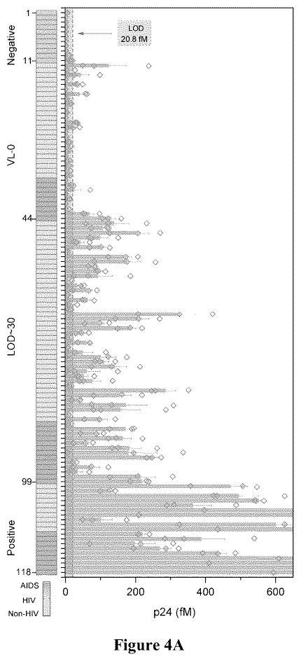

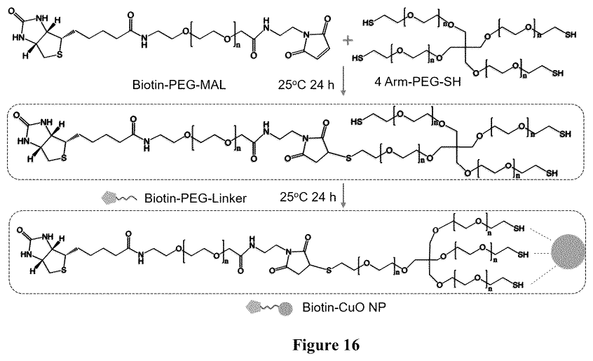

The use of the biotin-streptavidin linker was further investigated in the assay to increase the amount of CuONPs binding to each p24 antigen from 1 to 4 in order to lower the LOD (FIG. 3A). Two sandwich structures with (route II) and without (route I) the biotin-streptavidin linkers were formed in p24 spiked human sera. In route II, CuONPs were linked to detection antibodies using a streptavidin-biotin-polyethylene glycol (PEG) complex through copper-thiol adsorption chemistry. The biotin-PEG linker was synthesized in-house with commercially available reagents through addition reaction between the double bond in maleimide and the sulfhydryl (FIG. 16). After magnetic separation and acid treatment, Cu ion quantification by inductively coupled plasma-optical emission spectroscopy (ICP-OES) indicated that, with similar amount of Fe ions, the sample from route II contains ˜4× higher amount of Cu ions than the route I sample (FIG. 3B). Furthermore, transmission electron microscopy (TEM) and dynamic light scattering (DLS) results confirmed stable aggregation of CuONPs through biotin-streptavidin linkage in route II (FIGS. 3C and 3D). The synthesis of PEG linkers for linking CuONPs and biotins was characterized by NMR. According to ¹H NMR spectra (FIG. 3E), disappearance of the olefinic signal of the maleimide moiety in at δ6.7 ppm strongly corroborated the coupling reaction between Biotin-PEG-Maleimide and 4-Arm-PEG-Thiol, and the presence of biotin signals (δ3.9 and δ4.5 ppm) and residual thiol signal (δ2.6 ppm) confirmed the production of the final PEG-Biotin linkers, which will enable the conjugation of CuONPs and streptavidinized antibody. Fourier-transform infrared (FTIR) spectra with two absorption bands at 2866 cm⁻¹ and 1700 cm⁻¹ attributed to stretching bonds of C—H and C═O in PEG and biotin on CuONP surface, together with a weight loss of approximately 52% at 400° C. (FIG. 3F), confirmed the immobilization of PEG-Biotin linkers onto the CuONP surface.

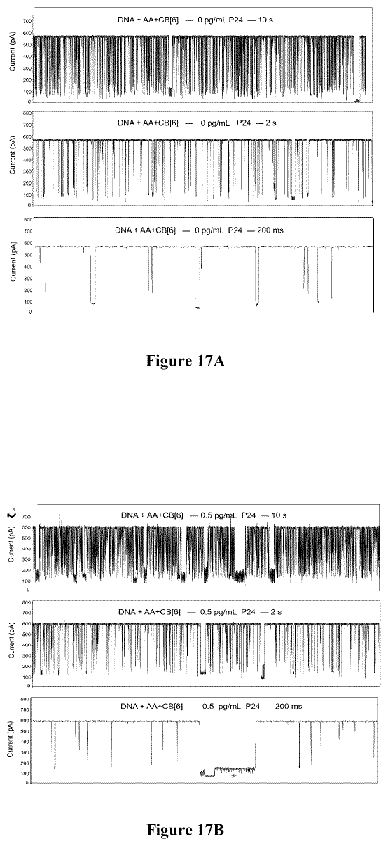

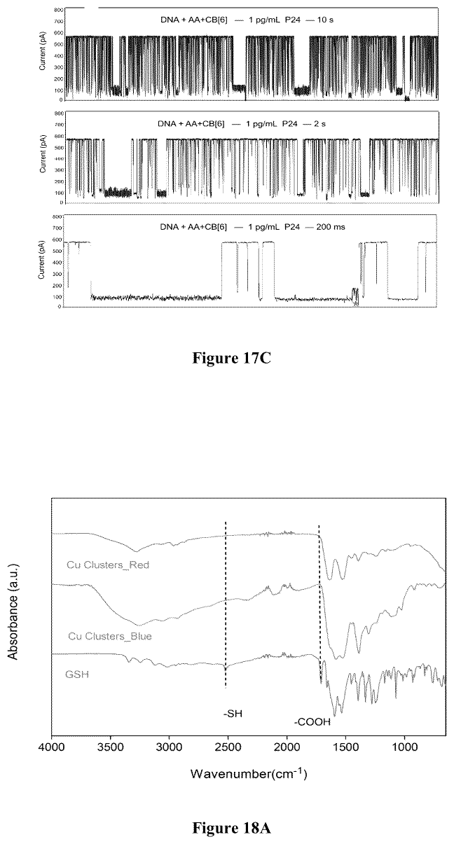

Next, CAN assays constructed by both synthesis routes were used to establish calibration curves using healthy donors' sera spiked with various amounts of recombinant p24 antigens (FIG. 3G). A positive correlation between multi-level event frequencies and p24 concentrations were observed in both assays (inset of FIG. 3G, wide range). However, in comparison with route I in which accuracy and reproducibility were greatly affected by low signal frequency, the biotin-streptavidin amplification strategy (route II) greatly improved detection accuracy and reproducibility, especially in the low p24 concentration range (0-42 fM), to achieve approximately 20-fold lower LOD than that of route I assay (inset of FIG. 3G, lower range) while reliably quantify changes in p24 concentration (Table 1). The Evaluation of Detection Capability for Clinical Laboratory Measurement Procedures (EP17 protocol) was used to further verify the LOD of the route II assay, which was determined using the limit of blank (LOB) and standard deviation (SD) of replicate tests of a sample known to contain a low concentration of analyte. Calculation of the LOB and the LOD follows previously established equations: LOB=mean_{blank}+1.645(SD_{blank}), LOD=LOB+1.645SD_{low concentration sample}). The calculated LOD (0.44 pg/mL, 18.3 fM) agrees with our calibration curve. An inspection of the raw current trace data recording also confirmed that multi-level signature events can be reliably detected in the 0.5 pg/mL (20.8 fM) but not in the blank control sample (FIG. 17).

TABLE 1
Capture rate of multi-level signal obtained from parallel
experiments for various p24 concentration in human serum.

| | p24 | p24 | Capture Rate (min⁻¹) | | | | | Recording | Counted |
|---|---|---|---|---|---|---|---|---|---|
| Route | pg/mL | pM | Pore 1 | Pore 2 | Pore 3 | Pore 4 | Pore 5 | Time (s) | Events |
| Route | 0 | 0 | 0 | 0.83 | 0.6 | 0.6 | 0 | 281 | 2 |
| II | 0.5 | 0.021 | 2.65 | 2.35 | 2.26 | 2.2 | 2.1 | 579.2 | 22 |
| | 1 | 0.042 | 2.78 | 3.79 | 3.03 | 2.78 | 2.74 | 926 | 47 |
| | 10 | 0.417 | 4.2 | 5.43 | 5.78 | 5.88 | 5.71 | 1252 | 113 |
| | 100 | 4.167 | 9.67 | 10.13 | 10.1 | 9.86 | 8.58 | 781 | 126 |
| | 1000 | 41.667 | 17.25 | 17 | 18.9 | 15.8 | 17.24 | 741.4 | 213 |
| Route | 0 | 0 | 0 | 0 | 0.79 | 0.88 | 0.6 | 452.2 | 3 |
| I | 0.5 | 0.021 | 0 | 0.69 | 0 | 0 | 1.29 | 356 | 2 |
| | 1 | 0.042 | 0 | 0.66 | 1.45 | 0 | 0 | 588.7 | 4 |
| | 10 | 0.417 | 2.77 | 3.03 | 2.96 | 2.7 | 3.57 | 427 | 22 |
| | 100 | 4.167 | 7.22 | 5.92 | 6.33 | 6 | 7.4 | 693.5 | 76 |
| | 1000 | 41.667 | 10.52 | 12 | 13.8 | 9.77 | 11.11 | 540.5 | 103 |

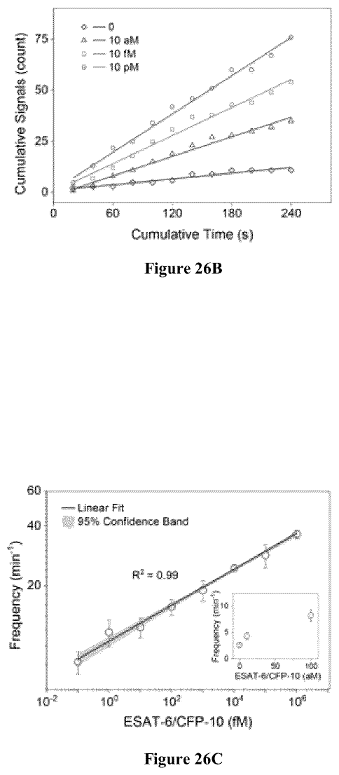

To increase cost effectiveness, the assay for the usage of capture and detection antibodies was optimized. 2.5 μg per ˜5×10⁷ dynabeads and 6 μg per 80 μg CuO particles, respectively, was selected for future experiments. To decrease the sampling volume demand for children, the lowest serum sample volume needed for each test to achieve the highest sensitivity was 100 μL. To achieve the shortest sample-to-answer time while maintaining highest reaction efficiency, both click reaction time and host-guest interaction time were optimized to 6 hours. In testing healthy sera spiked with various amounts of ESAT-6/CFP-10 antigen complex, raw current traces indicated a positive correlation between the oscillation signal frequency and the biomarker concentration (FIG. 26A). Cumulative counting of oscillation signals in 20 s intervals for each concentration showed steady increases, indicating that recording time between 1 to 4 minutes should yield consistent signal frequency can ensure reliable antigen quantification (FIG. 26B). Finally, a standard curve was established between oscillation signal frequency and ESAT-6/CFP-10 antigen complex concentration. The linear range of the curve indicates a limit of quantification (LOQ) of 100 aM and a large dynamic range up to 1 nM (FIG. 26C). Further examination of raw data confirmed that oscillation signals could be reliably detected in the 10 aM sample but not in the blank control sample, indicating that the assay achieved 10 aM limit of detection (LOD) (FIG. 26C). For comparison, an ELISA protocol was developed and optimized. However, its high LOD could not support ESAT-6/CFP-10 detection in clinical samples. Specificity validation was conducted by spiking excessive clinical interferents such as hemolysate, triglycerides, bilirubin, and human albumin into human serum. Individual interferents did not cause significant influence on nanopore test results. However, signal frequency was significantly dampened when the serum sample was contaminated with the combination of all interferents.

Analytical Performance Benchmarking.

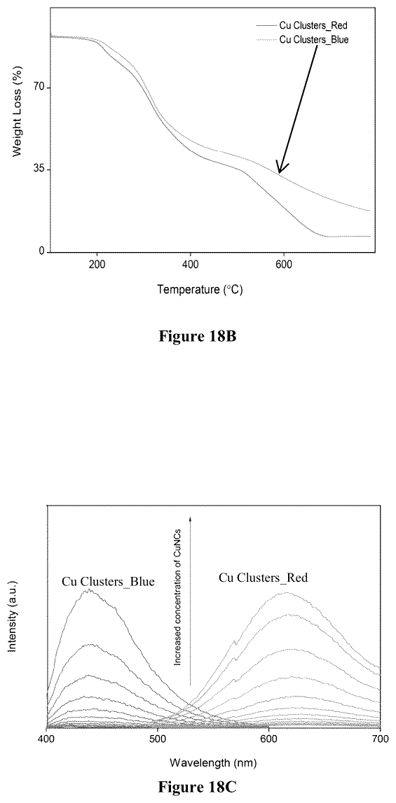

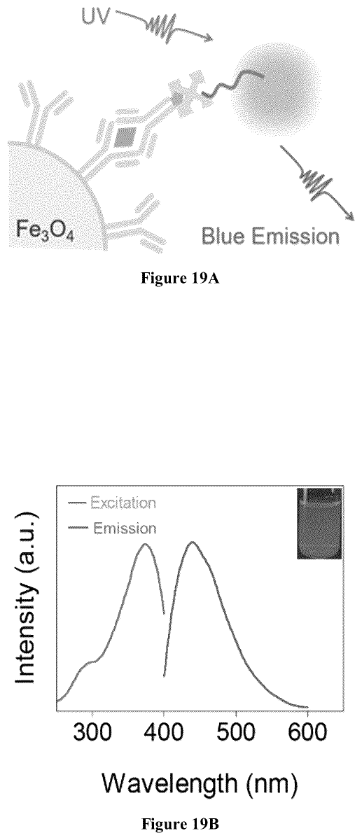

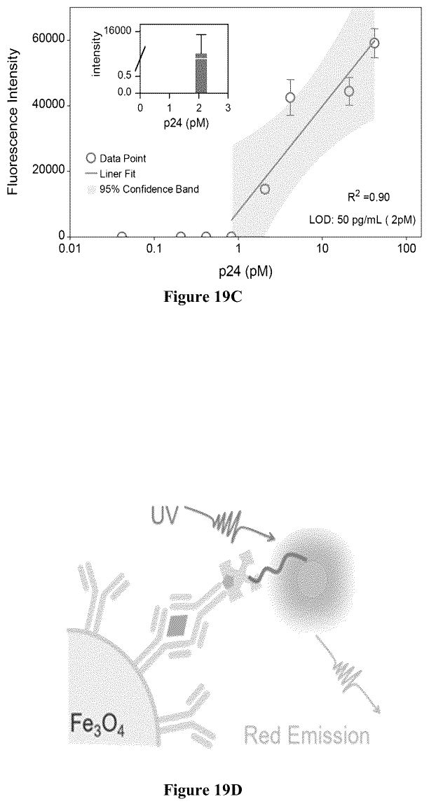

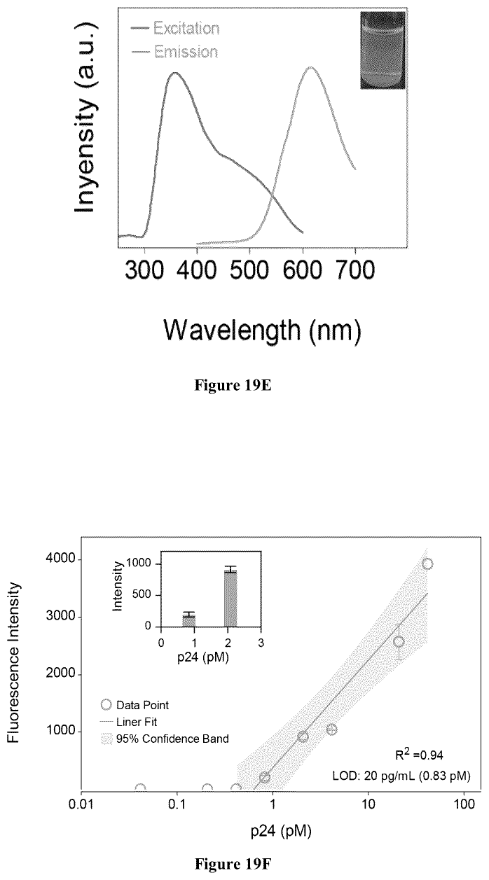

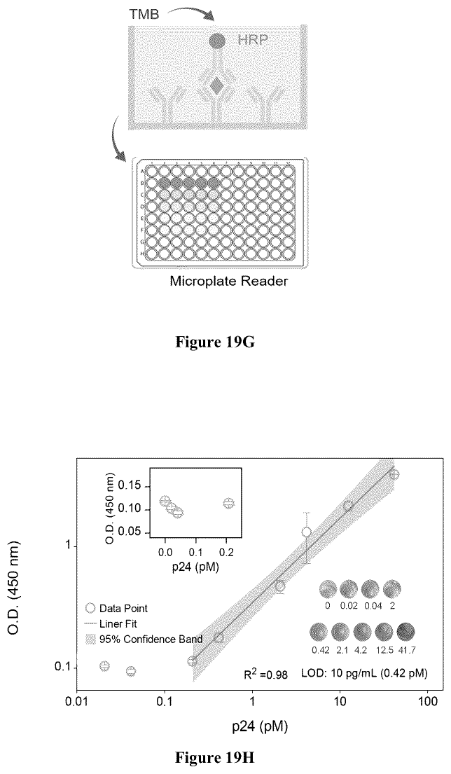

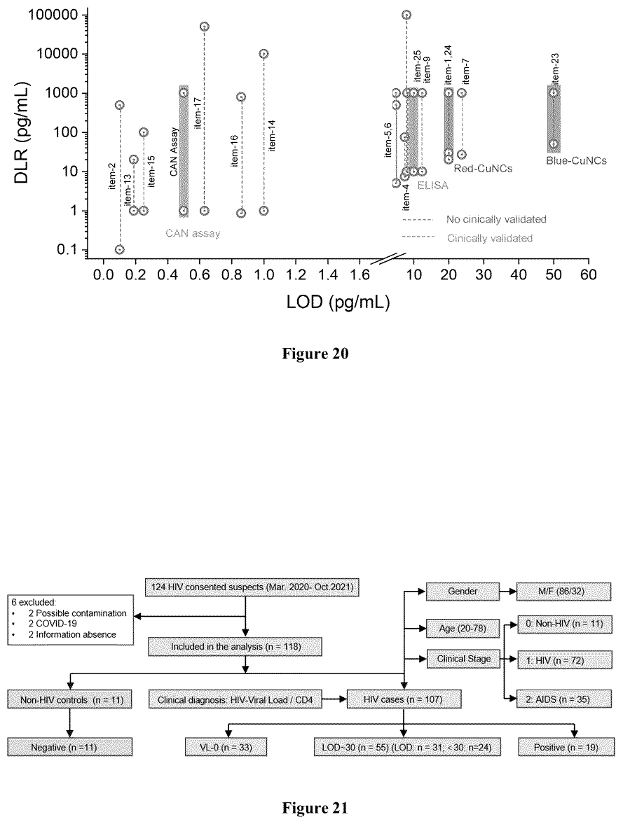

Recently, various approaches ranging from NP-based HIV biosensors to advanced ELISAs have been developed to detect p24 in human serum, but most of them are yet under diverse clinical studies. For comparison, a newly developed fluorescent nanocluster-based method and a traditional ELISA were performed side-by-side with the CAN assay disclosed herein. In the fluorescent nanocluster-based method, two types of glutathione (GSH) functionalized CuNCs with blue and red fluorescence were synthesized, characterized, and used to detect p24, respectively. Their simple chemical compositions allow easy engineering and optimization. By conjugating fluorescent CuNCs to the end of an antibody-antigen-antibody complex, p24 antigens can be detected using the fluorescence from CuNCs (FIG. 19A and 19D). Photoluminescence spectra and excitation spectra of these two types of GSH stabilized CuNCs indicate excitation at 370/360 nm and emission at 440/630 nm in aqueous solution, respectively (FIG. 19B and 19E). Positive correlation between the photoluminescence intensity and the concentration of CuNCs suggest that p24 antigens can be quantified by measuring the fluorescence intensity (FIG. 18C). Upon establishing calibration using p24 spiked sera, analytical LODs for the blue and the red fluorescent CuNCs assays were found to be 50 pg/mL (2.1 pM) and 20 pg/mL (0.83 pM), respectively (FIGS. 19C and 19F). Higher analytical sensitivity of the red fluorescent assay can be attributed to lower interference of background fluorescence. In another paralleled control experiment, an ELISA based fourth generation HIV-1 testing kit was able to reliably detect p24 at 10 pg/mL (0.41 pM), demonstrating 2-5-fold higher analytical sensitivity than CuNCs based assays (FIGS. 19G and 19H). Additional comparisons of LOD, dynamic range, and clinical validation with more techniques in previous reports were also summarizes to further highlight the superiority of the CAN assay (FIG. 20 and Table 2).

TABLE 2
Limit of detection (LOD) and dynamic linear range (DLR) comparison
of CAN assay to existing immunoassays.

| | | LOD | LOD | DLR | Clinically |
|---|---|---|---|---|---|
| Item | Assay method | (pg/mL) | (fM) | (pg/mL) | Validated |
| 1 | Carbon dot-based microplate and | 20 | 833 | 30-1000 | Y |
| | microfluidic chip immunoassay | | | | |
| 2 | Gold nanoparticle-based bio barcode | 0.1 | 4.2 | 0.1-500 | N |
| | amplification assay | | | | |
| 3 | Fluorescent silver nanoparticle-based | 8.2 | 342 | 10-1000 | Y |
| | immunoassay | | | | |
| 4 | Inductively coupled plasma mass | 7.5 | 312.5 | 7.5-75 | N |
| | spectrometry-based gold | | | | |
| | nanoparticle | | | | |
| | immunoassay | | | | |
| 5 | Gold nanocluster immunoassay | 5 | 208.3 | 5-1000 | Y |
| 6 | Europium nanoparticle-based | 5 | 208.3 | 5-500 | N |
| | microtiter-plate | | | | |
| 7 | Copper nanocluster based | 23.8 | 991.7 | 27-1000 | Y |
| | immunosensor | | | | |
| 8 | Europium nanoparticle sandwich | 0.19 | 7.91 | 1-20 | N |
| | immunoassay | | | | |
| 9 | Conventional HIV-1 p24 ELISA | 10-15 | 416-625 | 10-1000 | Y |
| 10 | Fourth-generation HIV screening | 4-10 | 166-416 | 5-1000 | Y |
| | immunoassays | | | | |
| 11 | Mercapto succinic acid hydrazide | 200 | 8333 | 500-500000 | N |
| | copper (II) (CuL) monolayer | | | | |
| | modified gold electrode (Au\|CuL) | | | | |
| | based immunosensor | | | | |
| 12 | Acetone-extracted propolis based | 6.4 | 266.7 | 10-60000 | N |
| | amperometry immunosensor | | | | |
| 13 | Immunosensor based on directly | 8 | 333.3 | 10-100000 | N |
| | electroplating an electrode surface | | | | |
| | with gold nanoparticles using | | | | |
| | chronoamperometry, | | | | |
| 14 | P-RGO@Au@Ru-SiO₂ composite | 1 | 41.7 | 1-10000 | N |
| | based electrochemiluminescence | | | | |
| | immunosensor | | | | |
| 15 | Selective detection of Ag⁺/ | 0.25 | 10.4 | 1-100 | Y |
| | Ag NPs and Cu²⁺/Cu⁺ using QDs | | | | |
| 16 | Surface-modified mesoporous | 0.86 | 35.8 | 0.86-800 | N |
| | nanofiber | | | | |
| 17 | Liposome-amplified | 0.63 | 26.25 | 1-50000 | N |
| | photoelectrochemical immunoassay | | | | |
| 23 | Blue emission fluorescent CuNCs | 50 | 2083 | 50-1000 | N |
| | linked immunosorbent assay | | | | |
| 24 | Red emission fluorescent CuNCs | 20 | 833 | 20-1000 | N |
| | linked immunosorbent assay | | | | |
| 25 | ELISA based fourth generation HIV- | 10 | 416.6 | 10-1000 | Y |
| | 1 test | | | | |
| 26 | Click chemistry amplified nanopore | 0.5 | 20.8 | 0.5-1000 | Y |
| | (CAN) assay | | | | |

CAN Assay Clinical Validation in a Pilot Cohort.

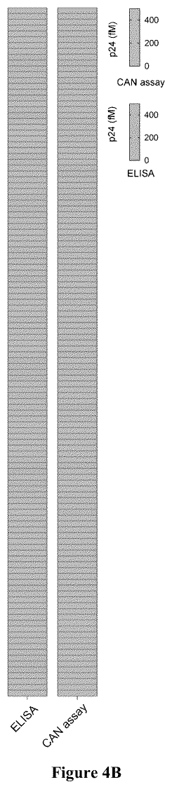

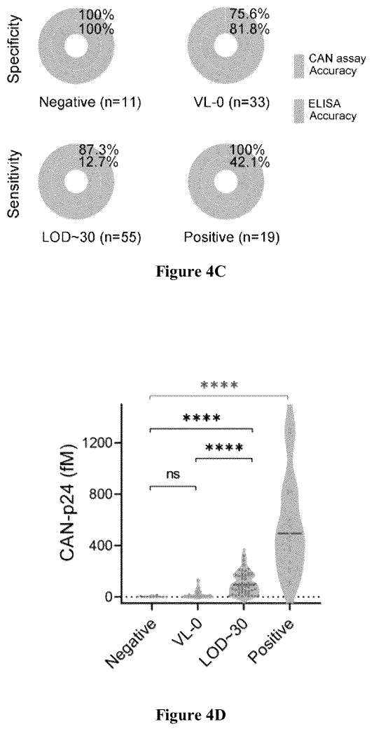

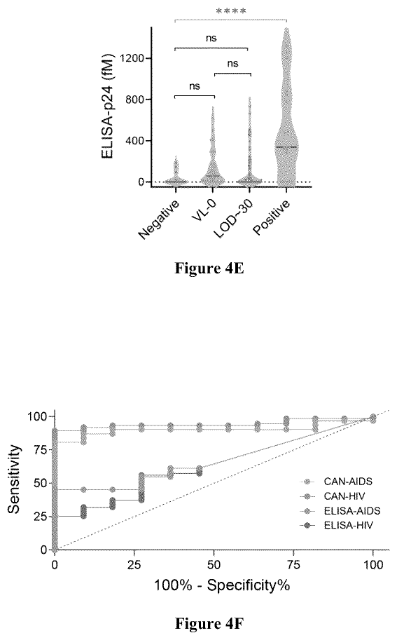

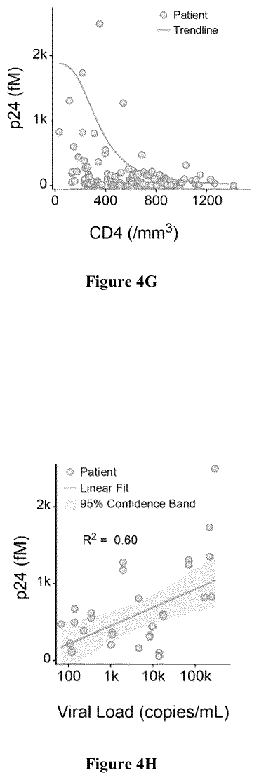

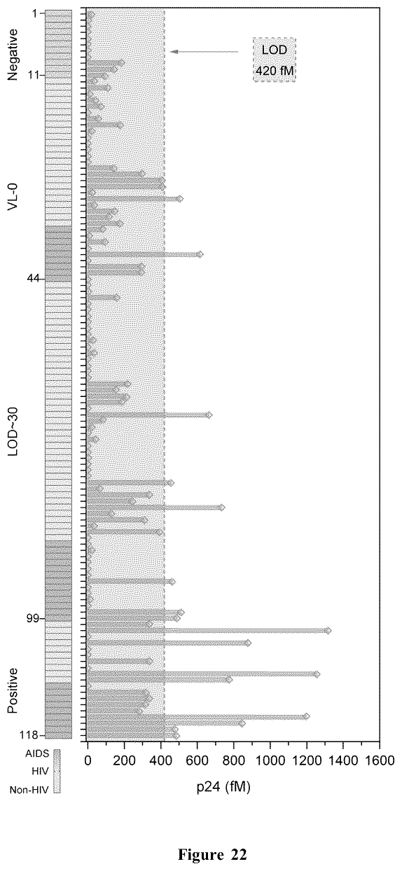

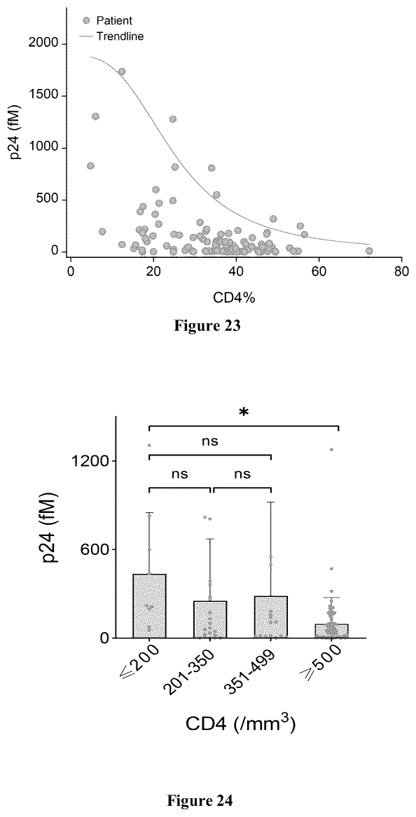

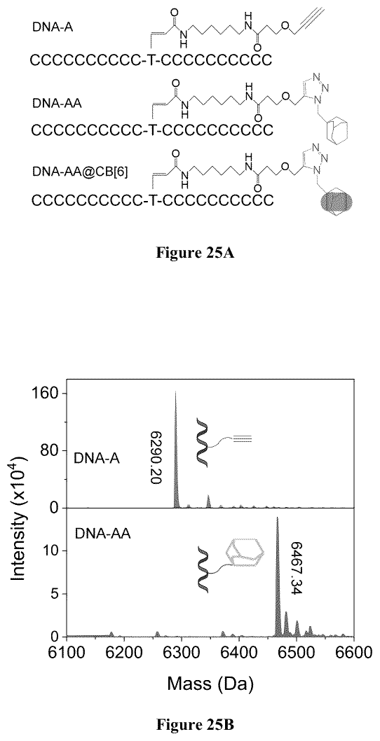

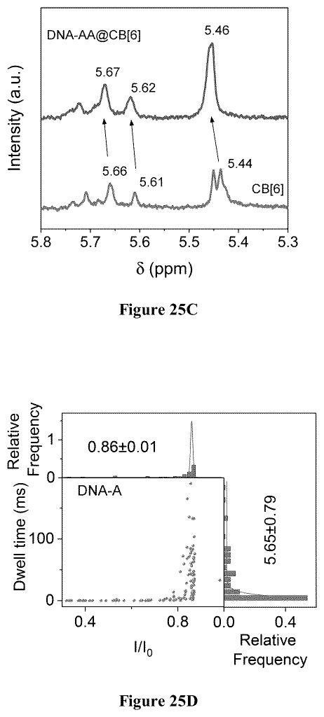

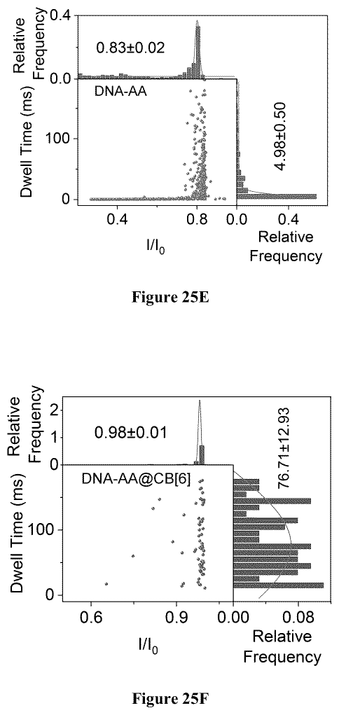

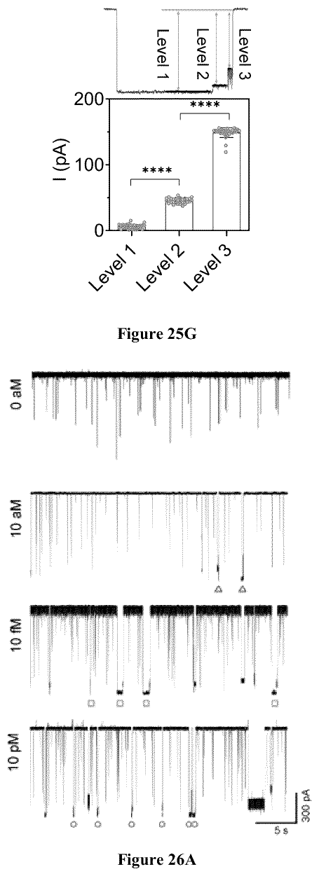

Among all methods experimented, the optimized CAN assay exhibited superior analytical sensitivity. Its clinical performance was further evaluated using 124 human samples (118 eligible) from a pilot HIV cohort. The ELISA based fourth generation HIV-1 test was also evaluated as a reference. Flow diagram describes the disposition of the study subjects (FIG. 21). Based on viral load results obtained at the same time of the sera collection, subjects were divided into four groups: Negative group (clinical state: Non-HIV), VL-0 group (HIV/AIDS stage with undetectable viral load), LOD-30 group (HIV/AIDS stage with viral load between LOD and 30 copies/mL), and Positive group (HIV/AIDS stage with >30 copies/mL viral load). Each sample was tested by the CAN assay and a commercialized fourth generation HIV-1 ELISA testing kit (FIGS. 4A and 22). While CAN results in general agree to ELISA results among subjects in the Negative group, the CAN assay showed superior sensitivity that allows p24 detection in patients missed by nucleic acid and/or ELISA assays in VL-0 and LOD-30 groups. Particularly, in clinically diagnosed patients with detectable viral load (LOD-30 and Positive groups), the CAN assay was able to detect p24 in 48 of 55 samples (87.3%) and 19 of 19 samples (100%), respectively, while the sensitivity of ELISA for these two groups is only 12.7% and 42.1% (FIG. 4B and C). The sensitivity improvement and the quantification capacity of the CAN assay also enables potential prognostic uses. Statistical analysis among three groups (Negative, VL-0 group, and LOD-30) shows that p24 was at similarly low level in Negative and VL-0 groups, but was significantly higher in patients with detectable (LOD-30) viral load. Meanwhile, no significant difference was detected between p24 across these groups measured by ELISA. ELISA was only able to detect significantly higher p24 as CAN did in patients with high (positive) viral load when they were added to the analysis (FIGS. 4D and 4E). To further compare the sensitivity and the specificity of the CAN assay and ELISA, receiver-operating characteristic (ROC) curves were constructed (FIG. 4F) based on results from these two assays for diagnosing HIV and AIDS. In each ROC curve, a cut-off value was determined to maximize the sensitivity and the specificity comprehensively. The area under the curve (AUC) of CAN-HIV and CAN-AIDS curves were significantly higher than those of ELISA curves, i.e., 0.95±0.02 (CAN-HIV, P<0.0001) and 0.90±0.04 (CAN-AIDS, P<0.0001) comparing to 0.63±0.07 (ELISA-HIV, P=0.16) and 0.66±0.08 (ELISA-AIDS, P=0.10), respectively. Using 2.29 fM and 17.01 fM as cut-off values, the CAN assay achieved 89.47% and 80.65% detection sensitivity in HIV and AIDS cohorts, respectively, with 100% specificity. CD4 cell count/percentage is an important immunological marker that indicates stage of infection and informs antiretroviral therapy. In patients with a valid CD4 count/percentage, correlation observed between CAN measured p24 levels and CD4 counts/percentage agrees with previous reports (FIGS. 4G and 23). Among patients categorized by CD4 counts, average p24 level was significantly higher in the severe immunosuppression group (≤200/mm³ CD4) than in the normal CD4 count group (CD4≤500/mm³) (FIG. 24). In patients with quantifiable viral loads, p24 also positively correlated with viral load (FIG. 4H), in consistency with previous reports.

Nanopore Assay for Sensitive and Specific Detection of Mtb Antigen Complex

Nanopore sensors operate by monitoring changes to an electrical current as molecules pass through a protein nanopore. However, nanopore signals usually have poor specificity, being induced simply by single molecules blocking the pore current. Traditional signal analysis that solely depends on dwell time and blockade of each signal usually fails to correctly distinguish molecules in complex samples, such as clinical specimens. The target biomarker of this assay is the ESAT-6/CFP-10 heterodimer antigen in blood. Due to its large size, low charge, and low concentration in the complex serum sample, direct detection with a nanopore is challenging and expected to yield low accuracy. Therefore, an assay process to immunoprecipitate the antigen complex from serum was designed and converted it to ssDNA probes through a catalytic click reaction to increase the detection sensitivity. Meanwhile, specificity is greatly improved by utilizing the characteristic signal of a host-guest modification to the probes. The assay protocol involves five specific steps: (1) Enriching ESAT-6/CFP-10 antigen complexes from human serum samples using capture antibody (anti-ESAT-6/CFP-10) modified dynabeads; (2) Forming sandwich structures with detection antibody (anti-ESAT-6/CFP-10) modified copper oxide (CuO) nanoparticles; (3) Releasing by Cu⁺ by acid treatment; (4) “Clicking” ssDNA-alkyne (DNA-A) with azido adamantane using a Cu+ catalyst to obtain ssDNA-azido adamantane (DNA-AA); and (5) Generating the host-guest structure of ssDNA probes by adding cucurbit[6]uril hydrate (CB[6]). Translocation of the ssDNA probes through an α-hemolysin (α-HL) nanopore produces highly characteristic signature signals (i.e., oscillation signal) that are clearly distinguished from non-specific signals of DNA-A and DNA-AA. The oscillation signal frequency correlates with the amount of ssDNA probes and, thus, the concentration of the ESAT-6/CFP-10 antigen complex. This process engenders a versatile biosensing strategy by combining the nanopore's single-molecule sensitivity to ssDNA with catalytic amplification, resulting in a unique signal pattern that allows detection and quantitation with high confidence.

ESA T-6/CFP-10 Quantification in Pediatric Samples

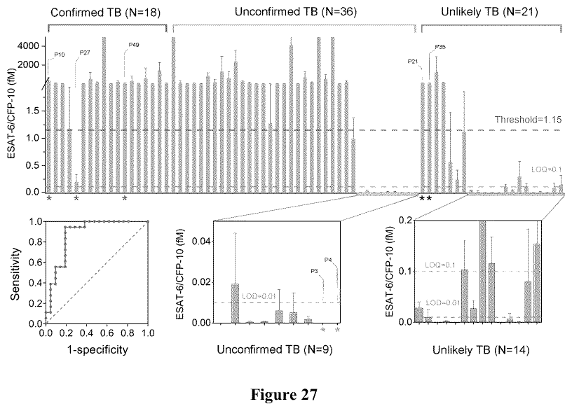

To assess the clinical performance of the nanopore assay, pediatric serum samples were collected and analyzed. Participants (N=75) were children, 3 months to 12 years old, with presumptive pulmonary TB who were enrolled for further TB evaluation and testing in a diagnostic study. The children were retrospectively categorized into ‘confirmed TB’, ‘unconfirmed TB’, or ‘unlikely TB’ groups, according to the NIH clinical case definitions and consideration of symptomatic responses to treatment (if treated) for TB (Table 3). After obtaining nanopore test results, the clinical classification was unblinded to the researchers for further analysis. The quantitative nanopore assay results obtained for all children are illustrated in FIG. 27. The mean ESAT-6/CFP-10 level±the standard deviation (SD) of three experiments for each child is depicted and grouped into one of the three categories (confirmed, unconfirmed, and unlikely TB). The graph shows cases with exceedingly low (between LOD and LOQ) and undetectable (below LOD) values in magnified insets under the main figure, for clarity. The diagnostic threshold value (1.15 fM) was determined using the receiver operating characteristic (ROC) analysis (FIG. 27) and was used to call if the result was positive (values above the threshold) or negative (values below the threshold) for TB by the nanopore assay. The optimal sensitivity and specificity values provided by the ROC analysis are 94.4% and 81.0%, respectively.

The number of positive (dark blocks) and negative (light blocks) diagnoses, as defined by the threshold value, determined from the nanopore assay, along with results from the clinical tests (TST, smear, Xpert, and Mtb culture) are illustrated in FIG. 27C for all children in each group (white blocks indicate children who did not receive a given test). Among the 18 children with confirmed TB, three (participant numbers: P10, P27, and P49, denoted by blue stars) had negative bacteriological results from baseline blood sample; these children were later confirmed for TB by positive Xpert tests performed on follow-up respiratory samples, collected between the first and second months of the study. All confirmed children began antituberculosis treatment at the time of baseline sample collection. The nanopore test results for this group were generally consistent with the results from clinical testing, with the exception of P27, who had ESAT-6/CFP-10 that was quantifiable (above the LOQ), but below the threshold. Notably, this child's chest x-ray (CXR) indicated TB and the child had a positive TST at baseline but was negative on all bacteriological tests (Mtb culture, Xpert, and smear).

TABLE 3

| | | | | |
|---|---|---|---|---|
| | Confirmed | Unconfirmed | Unlikely | All |
| Characteristic | TB^{γ} | TB^{Φ} | TB^{†} | participants |

Demographics

| | | | | |
|---|---|---|---|---|
| Male sex no./total no. (%) | 8/18 (44.44) | 21/36 (58.33) | 15/21 (71.43) | 44/75 (58.67) |
| Median Age (range) yr | 2 (0.58-8) | 2 (0.83-12) | 3 (0.25-10) | 2 (0.25-12) |

Clinical Characteristics (%)

| | | | | |
|---|---|---|---|---|
| Positive Xpert | 8/18 (44.44) | 0/36(0) | 0/21 (0) | 8/75 (10.67) |
| Negative Xpert | 10/18 (55.56) | 36/36 (100) | 21/21 (100) | 67/75 (89.33) |
| Positive Mtb Culture | 13/18 (72.22) | 0/36 (0) | 0/21 (0) | 13/75 (17.33) |
| Negative Mib Culture | 5/18 (27.78) | 36/36 (100) | 21/21 (100) | 62/75 (82.67) |
| Positive Tuberculin Skin Test* | 11/18 (61.11) | 9/36 (25.00) | 1/21 (4.76) | 21/75 (28.00) |
| Negative Tuberculin Skin Test | 3/18 (16.67) | 24/36 (66.67) | 17/21 (80.95) | 44/75 (58.67) |
| Significant TB Exposure^{#} | 8/18 (44.44) | 14/36 (38.89) | 3/21 (14.29) | 25/75 (33.33) |
| Well Defined Symptoms⁺ | 15/18 (83.33) | 33/36 (91.67) | 17/21 (80.95) | 65/75 (86.67) |
| TB Consistent Chest Radiograph | 12/18 (66.67) | 12/36 (33.33) | 2/21 (9.52) | 26/75 (34.67) |
| Positive Response to TB Treatment | 15/18 (83.33) | 22/36 (61.11) | 0/21 (0) | 37/75 (49.33) |

Nanopore Result (%)

| | | | | |
|---|---|---|---|---|
| Above LOQ | 18/18 (100) | 27/36 (75.00) | 11/21 (52.38) | 56/75 (74.67) |
| Above Threshold | 17/18 (94.44) | 26/36 (72.22) | 4/21 (19.05) | 47/75 (62.67) |

^{γ}Confirmed TB cases (positive Mtb culture or positive Xpert results for respiratory or stool samples).
^{Φ}Unconfirmed TB cases (if positive for ≥2 of the following criteria: significant TB exposure, positive tuberculin skin test, TB-associated symptoms, and abnormal TB-consistent chest radiograph.
^{†}Unlikely TB (if lacked two criteria for unconfirmed TB).
*Positive tuberculin skin test (the person's body was Infected with TB bacteria).
^{#}Significant TB exposure (index case = mother or household member, or someone spending >= 4 hours a day with child).
⁺Well defined symptoms of pediatric TB disease (2005).

Of the 36 children with unconfirmed TB, ten were designated as negative by the nanopore assay, with one quantifiable (above the LOQ), but below the threshold. Among these ten, two (P3 and P4, denoted by stars) should be redesignated as unlikely TB, as discussed in the following section. The remaining 26 children tested positive with above threshold ESAT-6/CFP-10 levels. The TSTs of 11 unconfirmed TB children were positive, eight of which coincided with positives from the nanopore test. None of the unconfirmed TB children tested positive on the smear, Xpert, or culture tests. The remaining 21 children were categorized as unlikely TB; four of these tested positive by the nanopore assay, two of which were deemed false-positives due to the absence of any clinical signs of the disease. It was concluded that the other two children, P21 and P35 (black stars), should be redesignated to the unconfirmed TB group, as discussed further in the following section.

Following NIH definitions, the nanopore assay's diagnostic sensitivity and specificity was calculated within the cohort. The 94.4% sensitivity for the confirmed TB group was significantly higher than that of the Xpert (44.4%) and Mtb culture (72.2%). In the unconfirmed TB group, the nanopore test detected TB in 26 of 36 children, revealing 72.2% of TB-positive children missed by the Xpert and Mtb culture methods. The 81.0% specificity of the nanopore test, though creditable, fell short of the 100% Xpert and culture specificity. However, it is accepted that the microbiological reference standard is imperfect in children and is likely to miss a proportion of children with TB disease, who have low bacillary loads which fall below the detection threshold for both culture and molecular tests. In terms of overall diagnostic performance, the nanopore test demonstrated enormous potential for the diagnosis of pediatric TB, outperforming current clinical technologies, including the TST, smear test, and the more sensitive Xpert, and Mtb culture.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method of detecting an antigen, the method comprising:
a. providing a sample comprising the antigen;
b. incubating the sample with a capture antibody-modified bead and a detection antibody-modified copper-based nanoparticle to form a sandwich structure complex;
c. separating the sandwich structure complex from the sample;
d. releasing copper ions from the separated sandwich structure complex in an acidic environment;
e. coupling a modified nucleic acid molecule and a second molecule comprising an azido functional group in the presence of the copper ions to form a polynucleotide complex, wherein the modified nucleic acid molecule is coupled to the azido functional group via a click reaction;
f. non-covalently bonding a probe to the polynucleotide complex to form a polynucleotide probe;
g. loading the polynucleotide-probe into a nanopore;
h. applying an electrical potential across said nanopore to induce translocation of the polynucleotide-probe through said nanopore; and
i. detecting an electrical signal associated with the translocation of said polynucleotide-probe through the nanopore;
wherein a characteristic of the signal is indicative of the presence or concentration of the antigen in the sample.

2. The method of claim 1, wherein the antigen is selected from the group consisting of a viral antigen, an infectious disease associated antigen, or a combination thereof.

3. The method of claim 2, wherein the infectious disease associated antigen is selected from the group consisting of an HIV associated antigen, a tuberculosis associated antigen, an influenza virus associated antigen, a meningitis associated antigen, or a combination thereof.

4. The method of claim 1, wherein the antigen comprises p24.

5. The method of claim 1, wherein the antigen comprises ESAT-6/CFP-10 antigen complex.

6. The method of claim 1, wherein the modified nucleic acid molecule comprises an alkyne modified DNA.

7. The method of claim 1, wherein the second molecule comprises 1-azidoadamantane.

8. The method of claim 1, wherein the probe comprises cucurbit[6]uril hydrate.

9. The method of claim 1, wherein the nanopore comprises an α-hemolysin nanopore.

10. The method of claim 1, wherein the non-covalent bond is independently selected from the group consisting of a hydrogen bond, an ionic bond, a van der Waals interaction, a hydrophobic interaction, a polar bond, a cation-pi interaction, a planar stacking interaction, and a metallic bond.

11. The method of claim 1, wherein the polynucleotide comprises double-stranded deoxyribonucleic acid (dsDNA), single-stranded DNA (ssDNA), peptide nucleic acid (PNA), single-stranded ribonucleic acid (ssRNA), DNA/RNA hybrid, or double-stranded ribonucleic acid (dsRNA).

12. The method of claim 1, wherein (d) further comprises: inserting the nanopore into a phosphate lipid bilayer wherein the phosphate lipid bilayer separates cis and trans compartments in an electrolyte solution.

13. The method of claim 12, wherein (e) further comprises: applying an external positive voltage to a trans facing side of the bilayer and grounding a cis facing side of the bilayer.

14. The method of claim 1, wherein the nanopore is less than 5 nm in diameter.

15. The method of claim 1, wherein the nanopore is less than 2 nm in diameter.
